(19) United States
(12) Patent Application Publication
(10) Pub. No.: US 2008/0226661 A1
(43) Pub. Date: Sep. 18, 2008

(54) Phage Screening Assay

(76) Inventors: John Bernard March, Midlothian (GB); Jason Clark, Midlothian (GB)

Correspondence Address: MYERS BIGEL SIBLEY & SAJOVEC, PO BOX 37428, RALEIGH, NC, 27627, US

(21) Appl. No.: 11/571,855

(22) PCT Filed: Jul. 11, 2005
(86) PCT No.: PCT/GB2005/002732

§ 371 Date: Nov. 15, 2007

(30) Foreign Application Priority Data
Jul. 9, 2004 (GB) 0415428.8

(51) Int. Cl. A61K 39/00; C12Q 1/70; A61K 35/66; A61K 38/16; A61P 31/04; A61K 31/7052; C12N 15/63; A61K 38/02

(52) U.S. Cl. 424/184.1; 435/5; 424/93.6; 514/2; 435/320.1; 514/12; 514/44

(57) ABSTRACT

The present invention relates to a method for identifying an agent for potential use in a vaccine. The present invention also provides agents identified by this method for use in a vaccine. The present further describes peptides of the pathogen Mycoplasma for use as a vaccine to treat or present diseases caused by Mycoplasma species, such as, Contagious bovine pleuropneumonia (CBPP).

### FIELD OF INVENTION

The present invention relates to a method for identifying an agent for potential use in a vaccine. The present invention also provides agents identified by this method for use in a vaccine. The present further describes peptides of the pathogen Mycoplasma for use as a vaccine to treat or prevent diseases caused by Mycoplasma species, such as, Contagious bovine pleuropneumonia (CBPP).

### BACKGROUND

Bacteriophages (or phages) are viruses of bacteria, which bind to specific receptors on their host cell and inject their DNA, which then either incorporates into the host genome (lysogeny) or is used to synthesise new phage particles which either extrude from the membrane without disrupting the cell (as with filamentous phage) or lyse the cell and release all the phage produced in the cell in a single burst (lytic growth). Phages can be single stranded DNA (such as the filamentous phages M13, fd and fl), double stranded DNA (e.g. the T series phages and λ phages), double stranded RNA (e.g. f6) or single stranded RNA (e.g. MS2 and Qβ).

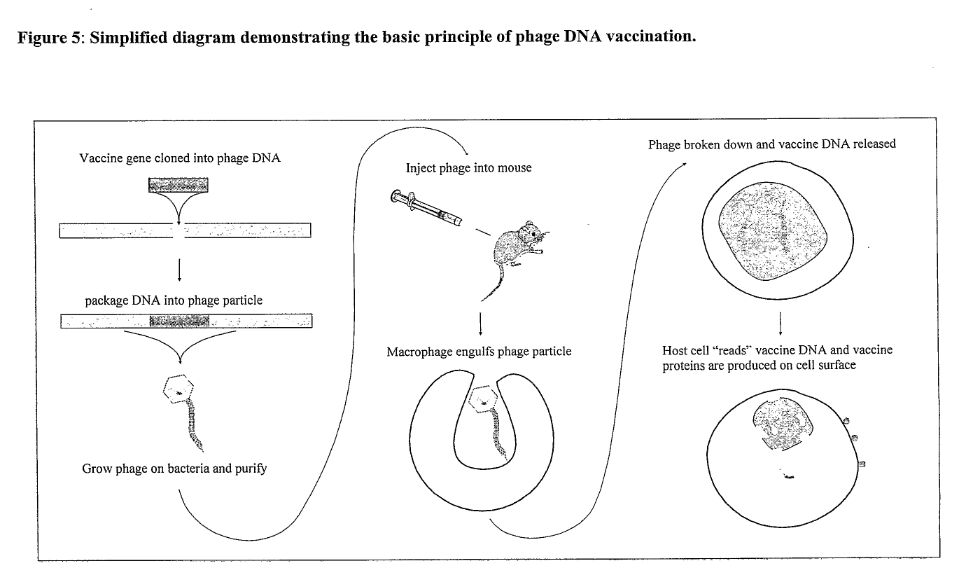

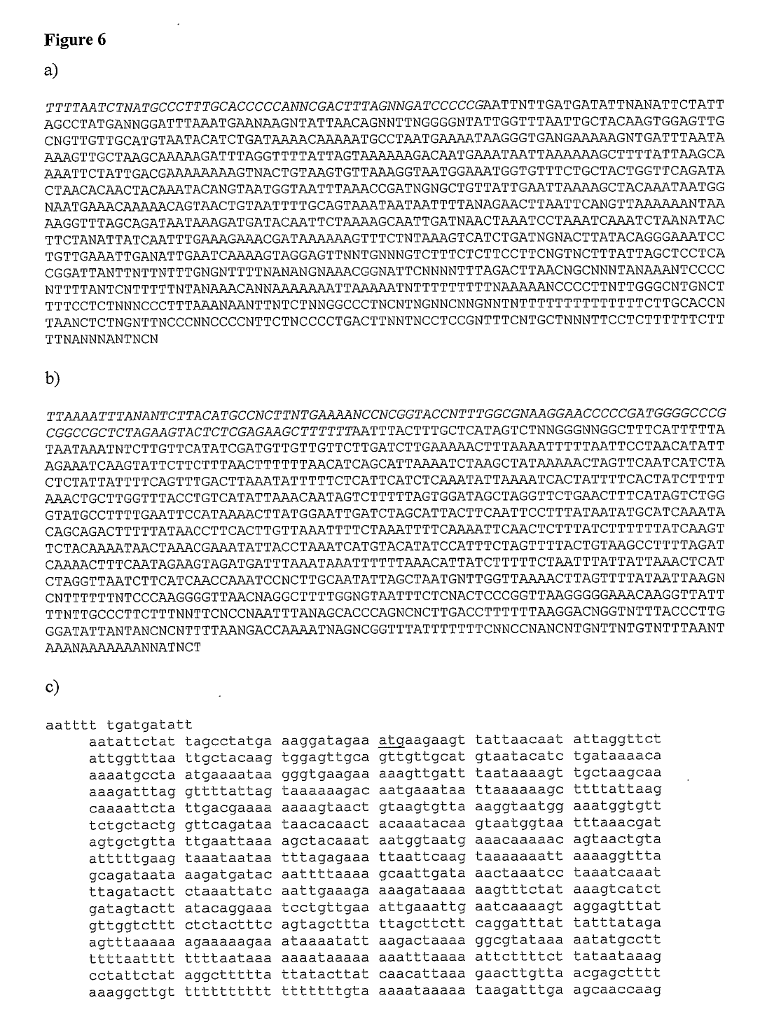

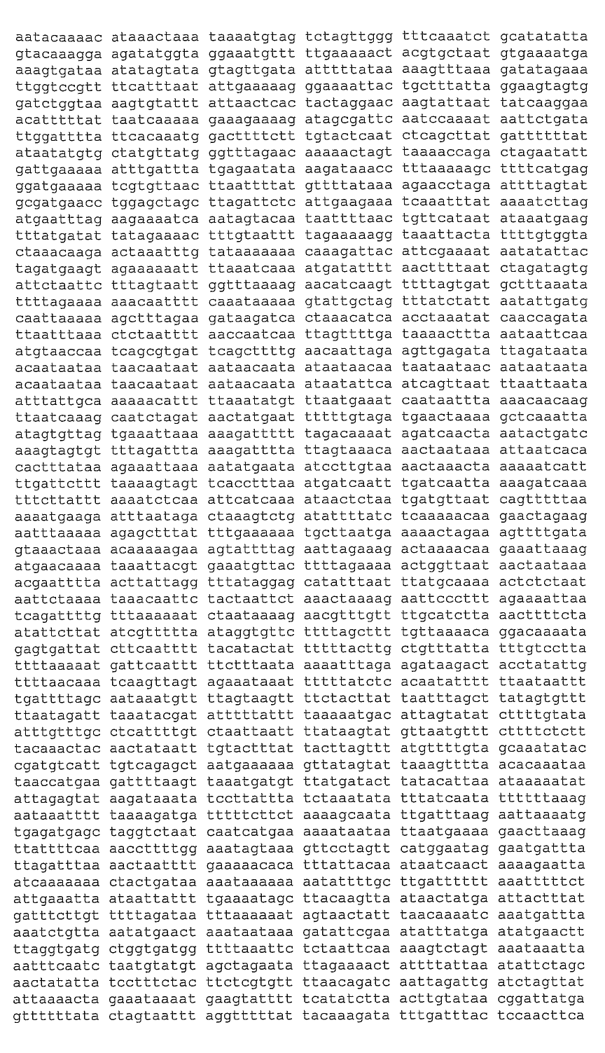

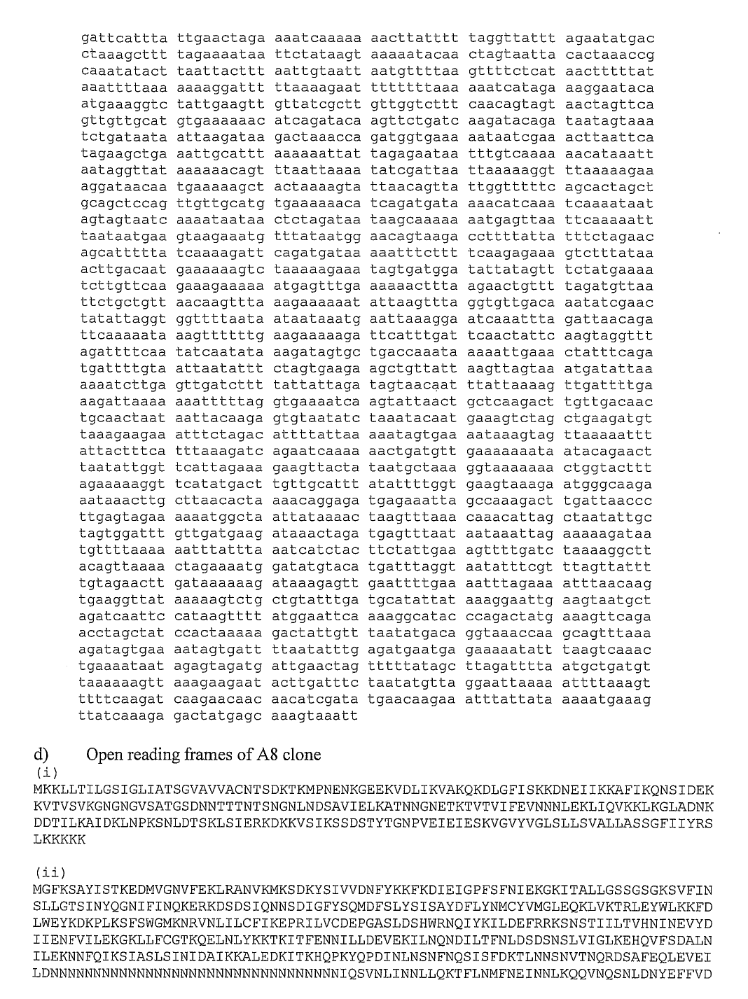

Recently, whole bacteriophage λ particles have been described as efficient delivery vehicles for administration of genetic or ‘DNA’ vaccines (rather than purified DNA as a vaccine) (Clark & March, 2004). This is in contrast to the procedure of DNA vaccination, wherein a vaccine gene is cloned into a eukaryotic expression cassette, consisting of a promoter and an upstream processing region, in a plasmid. The whole plasmid (“naked DNA”) is injected and the host raises an immune response to vaccine protein synthesised in vivo. DNA vaccination is particularly effective at inducing cytotoxic T-lymphocyte (CTL) responses (Leitner et al, 1999). due to intracellular expression of protein. However, in the phage DNA vaccination system, the gene encoding the vaccine antigen, under the control of a suitable eukaryotic promoter, is cloned into the bacteriophage genome and subsequently, the whole bacteriophage particle is used to inoculate the host for the purposes of raising a specific immune response against said vaccine antigen. In general, DNA vaccines are efficient at stimulating the cellular arms of the immune system (in particular CD8+ cytolytic T-cell responses) and are useful at overcoming maternal antibody and for producing conformationally active epitopes. This procedure is referred to here as Phage DNA Vaccination, and the basic principle is shown in FIG. 5.

There is, however, a constant need to identify and develop vaccines of defined antigenic profile for inoculation of subjects against known or less well-characterised/unknown pathogens, such as the causative pathogen of Severe Acute Respiratory Syndrome (SARS,) for example.

One such exemplary pathogen is Mycoplasma mycoides subsp. mycoides small colony type (MmmSC), the causative agent of contagious bovine pleuropneumonia (CBPP), an economically important disease of cattle currently affecting much of Africa and parts of southern Europe. Recent vaccination programmes based on freeze-dried cultures of the causative organism (MmmSC) have been unable to halt the re-emergence of the disease seen in recent years. Current OIE-recommended (O.I.E., 1996) vaccines (freeze-dried broth cultures of live attenuated MmmSC strains T₁44 or T₁ SR) exhibit relatively poor efficacy, and repeated vaccination is necessary to maintain protection (Thiaucourt et al., 2000). Vaccination can cause severe, sometimes fatal, reactions, leading to evasion by farmers. Other problems include the requirement for refrigeration in the field and the possibility of reversion to virulence (Rweyemamu et al., 1995, Hubschle 2003). Although it has been suggested that current live vaccines can be made considerably more stable and efficacious by following modifications to culture media and reconstitution procedures, it is apparent that a stable inexpensive and efficacious CBPP vaccine would offer many advantages, particularly if it offered a defined and recognisable antigenic profile, since this would allow the opportunity to differentiate between vaccinated and infected cattle, and would therefore be useful for control in Europe should the need arise.

Inactivated CBPP vaccines have been field tested, but results have generally been inconclusive, and detailed efficacy studies have not been performed (Garba et al., 1986). Successful immunization with an inactivated CBPP vaccine has been reported (Gray et al., 1986), but only when an extreme form of vaccination was used (two large doses of MmmSC in Freund's complete adjuvant). More recently, immunostimulating complex (ISCOM) protein sub-unit vaccines have been developed. Encouraging antibody responses were observed in vaccinated mice and cattle (including the induction of growth inhibiting antibodies); protection against CBPP field infection, however, has not yet been demonstrated (Abusugra et al., 1997; Abusugra and Morein, 1999). A major problem in designing an effective CBPP vaccine is the lack of understanding of the basic mechanism of immunity in cattle, although the apparent lack of correlation between antibody response and protection suggests that protection may be cell mediated, and the poor results with inactivated vaccines suggests that conformational epitopes may be important for protective efficacy.

It is amongst the objects of the present invention to obviate and/or mitigate at least one of the aforementioned disadvantages.

The present invention is based on the inventor's discovery of a technique using bacteriophage to screen and identify immunogenic polypeptides which may be suitable vaccine candidates. The present inventors have also identified vaccine candidates using the assay of the present invention for the prevention of contagious bovine pleuropneumonia (CBPP), a disease of cattle.

In a first aspect of the present invention there is provided a method for identifying a nucleic acid sequence or polypeptide for potential use in a vaccine, said method comprising:
a) providing a genetically modified phage comprising nucleic acid from a disease causing agent or diseased cell;
b) expressing a polypeptide(s) encoded by said nucleic acid sequence;
c) contacting said expressed polypeptide(s) with antiserum from an animal which has previously been infected with said disease causing agent and/or has said diseased cell(s); and
d) identifying said polypeptide(s) which specifically reacts with said antiserum.

It is understood that the invention may also be used to identify a phage comprising said nucleic acid and/or nucleic acid capable of expressing said polypeptide.

Typically, the phage is modified using standard molecular biology techniques so as to allow expression of said pathogen polypeptide(s) encoded by said pathogen nucleic acid sequence(s) by a phage infected host cell (see for example Sambrook et al, 2001). The phage may be designed using techniques known to those skilled in the art, to be capable of expressing the polypeptide(s) in a cell to be infected by said phage and/or to express the polypeptide(s) on the surface of the phage particle, by a technique known as phage display.

Typically nucleic acid such as genomic nucleic acid genome fragments may be cloned into the phage such that it is capable of being expressed by the phage once the phage transfects a target host cell which in the case of lambda phage is Escherichia coli.

Moreover, said phage may be engineered to express more than one polypeptide or antigen, as encoded by said nucleic acid. Typically, a known expression vector is used to express pathogen polypeptide(s) in said phage. For example, the cloning vehicles λ-gt11 or λ ZAP express may be used.

The polypeptide(s) encoded by said nucleic acid sequence are generally expressed by transfecting said phage into a host cell appropriate for each particular phage, so that the host cell's “machinery” will cause expression of the protein. Typically the phage will comprise appropriate transcription/translation signals for the given host cell, so that the cloned nucleic acid may be transcribed and the polypeptide thereafter translated and expressed.

Antiserum may be obtained from an animal which has previously been infected with the disease causing agent (live or dead), or comprises diseased cells, such as cancer cells, which may express polypeptides which a host would raise a humoral (antibody) immune response thereto. The antiserum will therefore comprise antibodies specifically reactive against antigens from said disease causing agent or diseased cells. Nevertheless, prior to carrying out the method according to the present invention, it would not be known to which antigens such antibodies have been raised against.

The animal may for example be a mammal such as a human, cow, sheep, pig, goat, rabbit, mouse or rat. Alternatively the animal may be a bird, such as a chicken, duck or turkey, or a fish, such as salmon, sea bass, trout or the like.

The antiserum is brought into contact with said expressed polypeptides. This may be achieved for example by first immobilising the phage to a substrate, such as nitrocellulose and lysing, if appropriate, the phage, in order to allow the polypeptide(s) to be exposed to the antiserum. The polypeptide may then be exposed or contacted with the antiserum, by for example washing the immobilised polypeptide(s) with a solution of neat or diluted antiserum.

If there are any antibodies present in the antiserum, which specifically react with an expressed polypeptide, such antibodies will bind to the polypeptide and this may be detected by, for example Western blotting techniques known in the art (see Sambrook et al, 2001) or, for example by way of a further labelled antibody, using techniques well known to those skilled in the art, such as ELISA, or radio-immunoassay (Sambrook et al, 2001).

A phage clone from which a positively reacting polypeptide has been identified may be subjected to a further round of screening to ensure/confirm the positive result.

It is also a straight-forward task to isolate the phage clone capable of expressing a positively reactive polypeptide(s) and to sequence the nucleic acid of the phage, so as to identify the cloned nucleic acid from the disease causing agent or diseased cell and consequently the expressed polypeptide and associated nucleic acid to which the antiserum has reacted. This may thereafter be used to check available databases to ascertain the identity of the polypeptide/gene if required.

A phage identified by the method according to the present invention containing nucleic acid and encoding an immunogenic polypeptide may be used directly to vaccinate a host animal (e.g. mammals, birds, fish, reptiles or amphibians) as described for example in WO/02076498. Alternatively, the polypeptide may be expressed by said phage, or nucleic acid encoding said polypeptide excised and cloned into another suitable vector and expressed thereby, such that the polypeptide may be purified and subsequently used in the provision of a so-called sub-unit vaccine.

Optionally, the phage of the present invention preferably further comprises appropriate transcription/translation regulators such as promoters, enhancers, terminators and/or the like for controlling expression of polypeptides in eukaryotic host cells. Typically the promoter may be a eukaryotic promoter such as CMV, SV40, thymidine kinase, RSV promoter or the like. Conveniently the promoter may be a constitutive promoter. However, controllable promoters known to those of skill in the art may also be used. For example constructs may be designed which comprise the exogenous (pathogen) nucleic acid under control of a constitutive promoter and a controllable promoter by way of cloning into an expression vector of choice. In this manner it may be possible to cause expression of the exogenous nucleic acid initially by way of the constitutive promoter and at a second time point by expression from the controllable promoter. This may result in a stronger immune response when following the techniques described in WO/0207/6498.

The term nucleic acid may refer to ribo- or deoxy ribo-nucleic acid (RNA or DNA, respectively). Conveniently, genomic DNA may be cloned into a vector of choice to be expressed in said phage. Preferably, a random or semi-random genome library of the genome of a disease causing agent or diseased cell of choice may be cloned into a phage vector under a promoter of choice. In this manner the nucleic acid of many, if not a majority of the polypeptides expressed by said disease causing agent or diseased cell may be cloned in order to allow the screening of suitable immunogenic polypeptides.

In general, the term “polypeptide” refers to a chain or sequence of amino acids displaying an antigenic activity and does not refer to a specific length of the product as such. The polypeptide if required, can be modified in vivo and/or in vitro, for example by glycosylation, amidation, carboxylation, phosphorylation and/or post translational cleavage, thus inter alia, peptides, oligo-peptides, proteins and fusion proteins are encompassed thereby. Naturally the skilled addressee will appreciate that a modified polypeptide should retain physiological function i.e. be capable of eliciting an immune response.

The term phage according to the present invention includes single stranded DNA phages (such as the filamentous phages M13, fd and fl), double stranded DNA phages (e.g. the T series phages and λ phages), double stranded RNA phages (e.g. f6) or single stranded RNA phages (e.g. MS2 and Qβ). Preferably, double stranded DNA phage λ is used, as such phage have the additional advantages of stability under high ambient conditions, ease and cheapness of production, together with providing a highly immunological signal (against the phage coat) which provides an easily assayed marker with which to check for vaccination.

In a second aspect of tie present invention there is provided use of an polypeptide(s) identified by a method according to the present invention for manufacture of a vaccine for prophylactic or therapeutic administration.

It is to be appreciated that according to the second aspect, the present invention is applicable to the preparation of a vaccine for practically any disease, such as those caused by a pathogen, providing that a suitable immuno-protective response can be raised to a protein or proteins of an infectious agent (pathogen).

The term pathogen according to the present invention encompasses virus, bacteria, fungi, yeast, protozoa, helminths, insecta, and transmissible spongiform encephalopathies, for example. The pathogens for which the agents of the present invention would be applicable to, include pathogen-causing infectious diseases of both humans and animals. Lists of suitable diseases are well known to those versed in the art and examples are to be found in the O.I.E. Manual of Standards and Diagnostic Tests 3rd Ed., OIE, Paris 1996, Topley & Wilson's Principles of Bacteriology, Virology and Immunity 8th Ed., Eds. Parker M. T. and Collier L. H., Vol IV (Index), Edward Arnold, London 1990, The Zoonoses: Infections Transmitted from Animals to Man. Bell J. C. et al., Edward Arnold, London 1988 and Parasitology: The Biology of Animal Parasites 6th Ed. Noble E. R. et al., Lea & Febiger, Philadelphia, 1989. In addition, agents identified by the present invention for use as a vaccine could be used to elicit an immune response against cancer cells by means of the expression of a cancer cell specific antigen as the immunogenic polypeptide.

The present invention further provides novel modified phage expressing peptides of the pathogen Mycoplasma, eg., Mycoplasma mycoides subsp. mycoides small colony type (MmmSC) for use as a vaccine to treat or prevent Contagious bovine pleuropneumonia (CBPP). Thus, in a yet further aspect of the present invention there is provided a modified phage(s) capable of expressing a polypeptide encoded by a nucleotide sequence A8, or functional fragment, homologue or derivative thereof for use in a vaccine for the prevention of disease caused by the pathogen MmmSC, wherein A8 comprises the nucleotide sequence as shown in FIG. 6c.

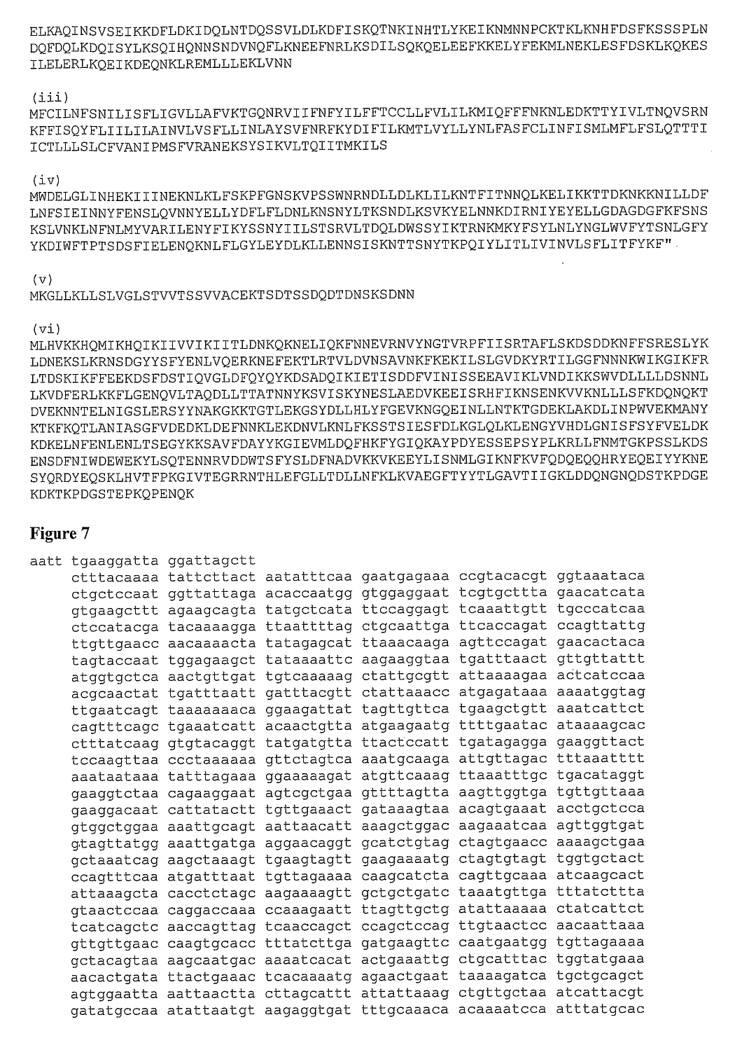

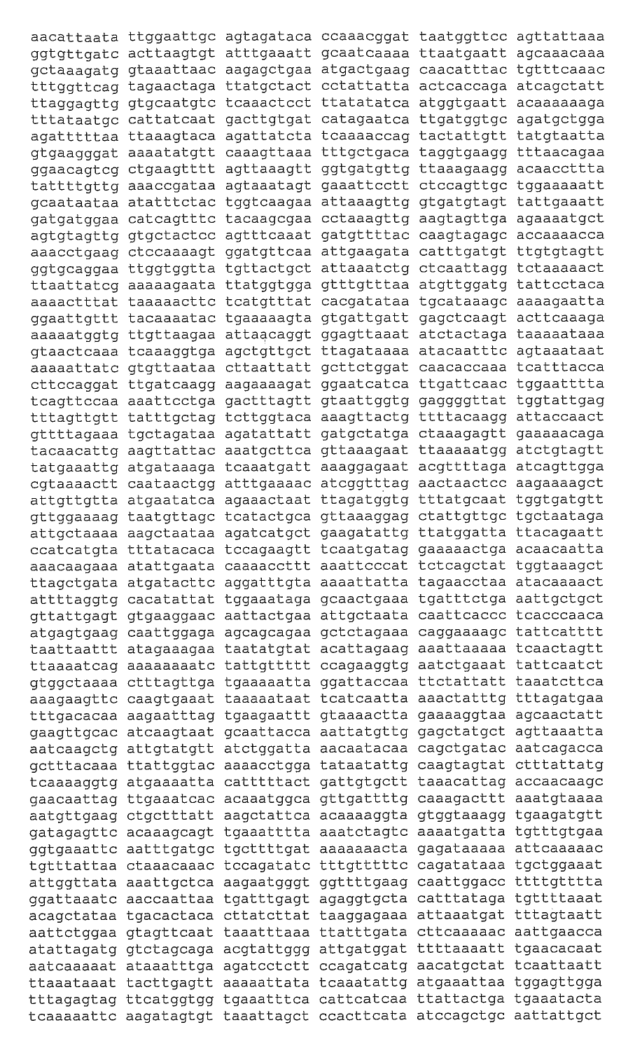

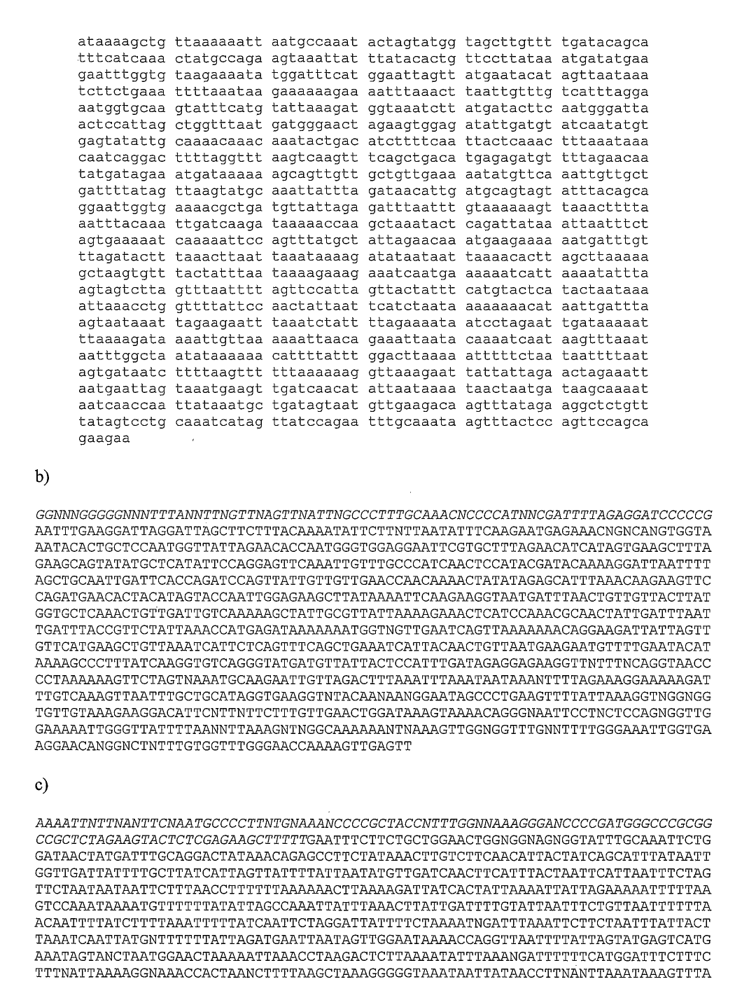

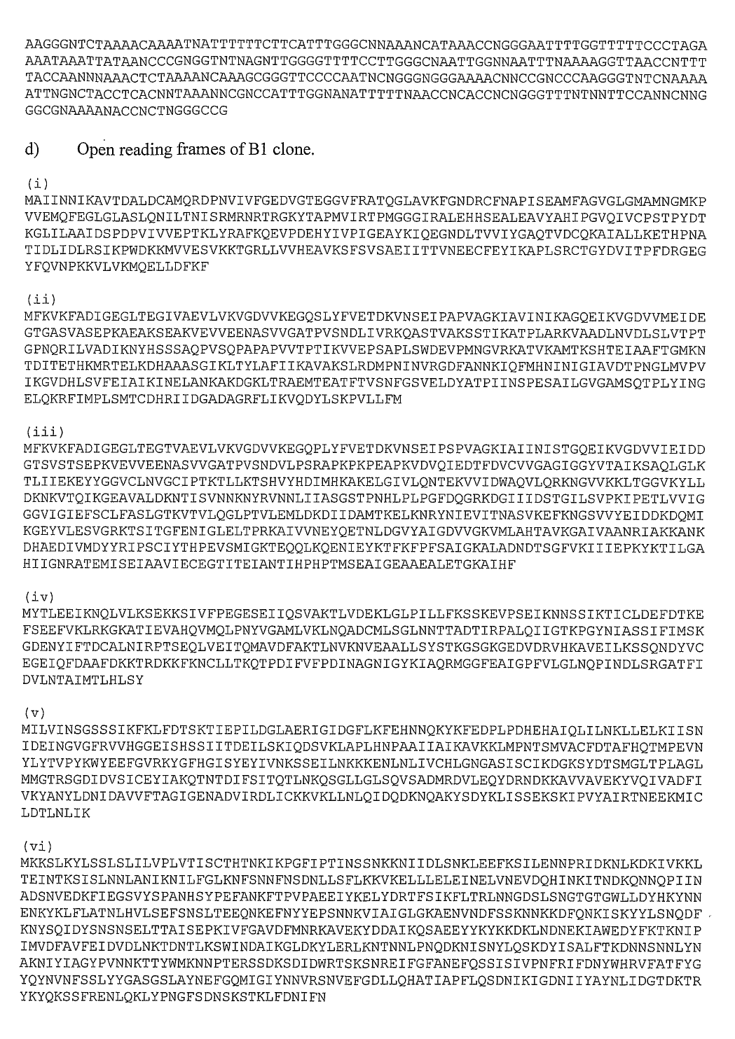

In a yet further aspect of the present invention there is provided a modified phage(s) capable of expressing a polypeptide encoded by a nucleotide sequence B1, or functional fragment, homologue or derivative thereof for use in a vaccine for the prevention of disease caused by the pathogen MmmSC, wherein B1 is the nucleotide sequence as shown in FIG. 7a.

In a yet further aspect of the present invention there is provided a modified phage(s) capable of expressing a polypeptide or functional fragment, homologue or derivative thereof for use in a vaccine for the prevention of disease caused by the pathogen MmmSC, wherein said polypeptide is at least one prolipoprotein as shown in FIGS. 6 (d) or 7 (d) or functional fragment, homologue or derivative thereof.

It should be understood that “functional fragment, homologue or derivative thereof” relates to nucleic acid or polypeptide sequences with a similar function. That is, nucleic acid sequences or polypeptides capable of effecting a suitable immuno-protective response to an infectious agent (pathogen).

Generally speaking, “homologue” relates to nucleic acid or polypeptide sequences sharing at least 25%, 50%, particularly 60, 70 and 80%, and especially 90 and 95% identity to the nucleic acid sequences as shown in FIGS. 6(c) or 7(a) or prolipoprotein amino acid sequences as shown in FIGS. 6(d) or 7(d). % sequence identity may be determined when the alignment or comparison is conducted by a computer homology program or search algorithm known in the art. By way of example and not limitation, useful computer homology programs include the following: Basic Local Alignment Search Tool (BLAST) (www.ncbi.nlm.nih.gov) [Altschul et al., (1990). The BLAST Algorithm. J. Mol. Biol., 215, 403-410.Altschul et al., (1997). Nuc. Acids Res., 25, 3389-3402.] a heuristic search algorithm tailored to searching for sequence similarity which ascribes significance using the statistical methods of Karlin and Altschul [Karlin and Altschul, (1990). Proc. Natl. Acad. Sci. USA, 87, 2264-2268. Karlin and Altschul, (1993). Proc. Natl/Acad. Sci. USA, 90, 5873-5877.]. Five specific BLAST programs perform the following tasks:

The BLASTP program compares an amino acid query sequence against a protein sequence database.

The BLASTN program compares a nucleotide query sequence against a nucleotide sequence database.

The BLASTX program compares the six-frame conceptual translation products of a nucleotide query sequence (both strands) against a protein sequence database.

The TBLASTN program compares a protein query sequence against a nucleotide sequence database translated in all six reading frames (both strands).

The TBLASTX program compares the six-frame translations of a nucleotide query sequence against the six-frame translations of a nucleotide sequence database.

Smith-Waterman (database: European Bioinformatics Institute www.ebi.ac.uk/bic_sw/) [Smith-Waterman, (1981). J. Mol. Biol., 147, 195-197.] is a mathematically rigorous algorithm for sequence alignments.

FASTA (see [Pearson et al., (1988). Proc. Natl. Acad. Sci. USA, 85, 2444-2448.] is a heuristic approximation to the Smith-Waterman algorithm. For a general discussion of the procedure and benefits of the BLAST, Smith-Waterman and FASTA algorithms see [Nicholas et al., (1998). A tutorial on searching sequence databases and sequence scoring methods. www.psc.edu.] and references cited therein.

The present invention further describes use of the phage expressing polypeptides encoding nucleotide sequences A8 and/or B1 of the present invention and/or lipoproteins as shown in FIG. 6d) and/or 7d) as described herein for the manufacture of vaccines for the prevention or treatment of Contagious bovine pleuropneumonia (CBPP) caused by the pathogen Mycoplasma mycoides subsp. mycoides small colony type (MmmSC., as well as a method of prophylactically treating CBPP using a nucleic acid and/or polypeptide(s) encoding a prolipoprotein or lipoprotein as shown in FIGS. 6d and/or 7d or functional fragment, homologue or derivative thereof. The vaccine potential of such phage X constructs carrying MmmSC nucleic acid sequences A8 and B1 are assessed herein, showing antibody responses and cellular proliferation in vaccinated mice following infection with MmmSC, and additionally measuring the length of the mycoplasmaemia in infected animals by means of a mouse infection technique (Smith 1965, 1969, 1971a,b).

In a preferred presentation, the vaccine can also comprise an adjuvant. Adjuvants in general comprise substances that boost the immune response of the host in a non-specific manner. A number of different adjuvants are known in the art. Examples of adjuvants may include Freund's Complete adjuvant, Freund's Incomplete adjuvant, liposomes, and niosomes as described in WO90/11092, mineral and non-mineral oil-based water-in-oil emulsion adjuvants, cytokines, short immunostimulatory polynucleotide sequences, for example in plasmid DNA containing CpG dinucleotides such as those described by Sato Y. et al. (1996); and Krieg A. M. (1996). Such adjuvants may in fact be expressed by the phage which is capable of expressing a polypeptide(s) identified by the method of the present invention.

Further adjuvants of use in the invention include encapsulators comprising agents capable of forming microspheres (1-10 μm) such as poly(lactide-coglycolide), facilitating agents which are capable of interacting with polynucleotides such that the said polynucleotide is protected from degradation and which agents facilitate entry of polynucleotides such as DNA into cells. Suitable facilitating agents include cationic lipid vectors such as:
1,3-di-oleoyloxy-2-(6-carboxy-spermyl)-propylamid (DOSPER),
N-[1-(2,3-dioleoyloxy)propyl]-N,N,N-trimethylammoniummethylsulfate (DOTAP),
N-[1-(2,3-dioleoyloxy)propyl)]-N,N,N-trimethylammonium chloride (DOTMA),
(N,N,N′,N′-tetramethyl-N,N′-bis(2-hydroxylethyl)-2,3-dioleoyloxy-1,4-butanediammonium iodide,
bupivacaine-HCl, non-ionic polyoxypropylene/polyoxyethylene block copolymers, polyvinyl polymers and the like.

Such cationic lipid vectors can be combined with further agents such as L-dioleoyl phosphatidyl ethanolamine (DOPE) to form multilamellar vesicles such as liposomes.

The mode of administration of the vaccine of the invention may be by any suitable route that delivers a suitable amount of the nucleic acid construct, or vector of the invention to the subject. However, the vaccine is preferably administered parenterally via the intramuscular or deep subcutaneous routes. Other modes of administration may also be employed, where desired, such as oral administration or via other parenteral routes, i.e., intradermally, intranasally, or intravenously.

Generally, the vaccine will usually be presented as a pharmaceutical formulation including a carrier or excipient, for example an injectable carrier such as saline or a pyrogenic water. The formulation may be prepared by conventional means. It will be understood, however, that the specific dose level for any particular recipient animal will depend upon a variety of factors including age, general health, and sex; the time of administration; the route of administration; synergistic effects with any other drugs being administered; and the degree of protection being sought. Of course, the administration can be repeated at suitable intervals if necessary.

The present invention further provides a vaccine formulation comprising nucleic acid and/or polypeptide sequences as described herein or identified by the methods described herein. Optionally the formulation may further comprise a suitable carrier therefore.

The present invention will now be described by way of example and figures as follows:

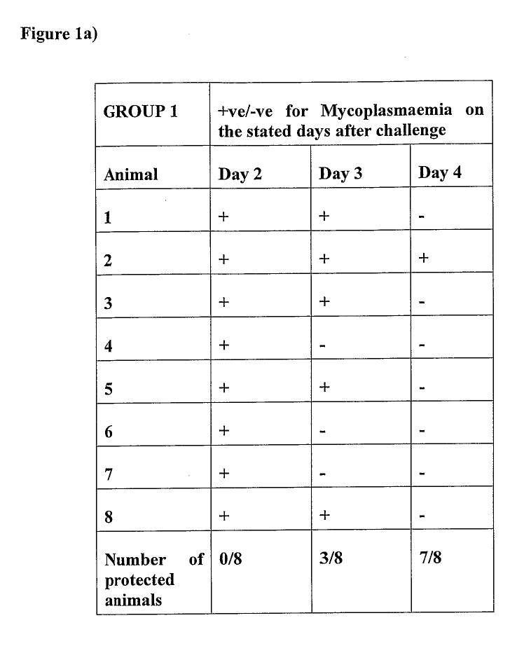

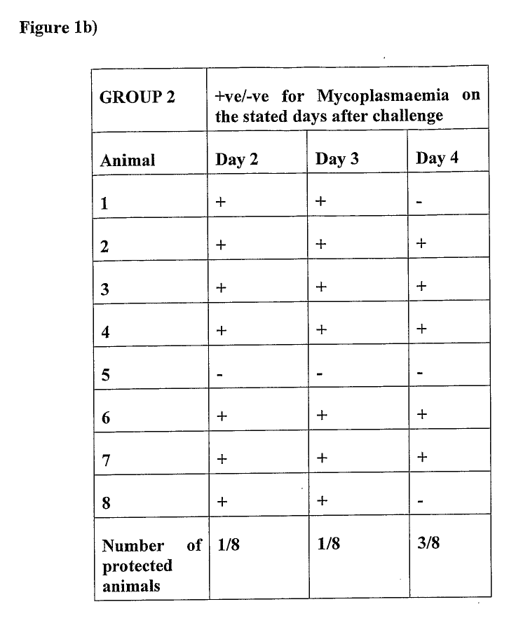

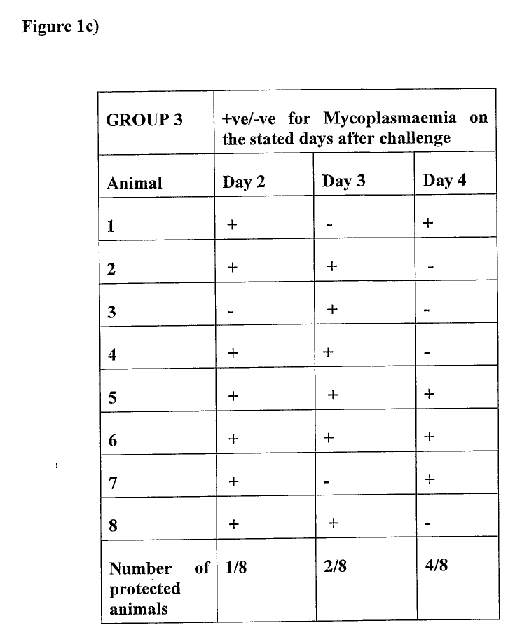

FIG. 1. Number of mice in each vaccinated group exhibiting mycoplasmaemia after intraperitoneal challenge

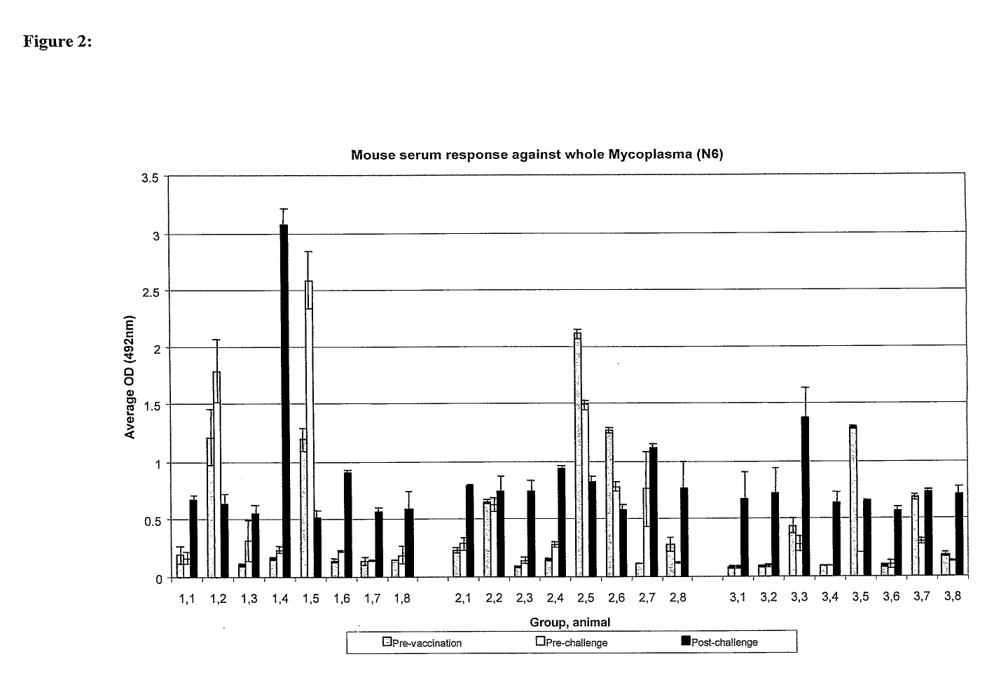

FIG. 2. Anti-Mycoplasma immune responses (measured by ELISA) in BALB/c mice before and after vaccination and pre and post challenge. Error bars are standard deviations of the mean OD_{492 nm} for each mouse on each separate bleed. Key: [symbol] pre-vaccination, [symbol] post-vaccination, [symbol] post-challenge.

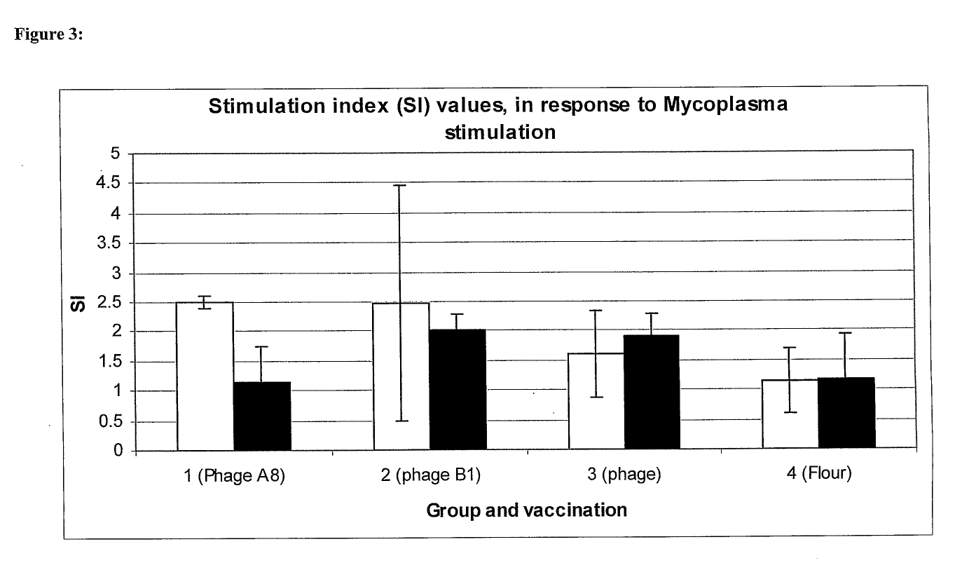

FIG. 3. Anti-mycoplasma (dead and alive) immune cell proliferative responses (measured by LSA) in BALB/c mice after challenge with whole Mycoplasma. Key: [symbol] Media (−ve control), [symbol] Live Mycoplasma, [symbol] Dead Mycoplasma.
N-[1-(2,3-dioleoyloxy)propyl]-N,N,N-trimethylammoniummethylsulfate (DOTAP),
N-[1-(2,3-dioleoyloxy)propyl)]-N,N,N-trimethylammonium chloride (DOTMA),
(N,N,N′,N′-tetramethyl-N,N′-bis(2-hydroxylethyl)-2,3-dioleoyloxy-1,4-butanediammonium iodide,
bupivacaine-HCl, non-ionic polyoxypropylene/polyoxyethylene block copolymers, polyvinyl polymers and the like.

Such cationic lipid vectors can be combined with further agents such as L-dioleoyl phosphatidyl ethanolamine (DOPE) to form multilamellar vesicles such as liposomes.

The mode of administration of the vaccine of the invention may be by any suitable route that delivers a suitable amount of the nucleic acid construct, or vector of the invention to the subject. However, the vaccine is preferably administered parenterally via the intramuscular or deep subcutaneous routes. Other modes of administration may also be employed, where desired, such as oral administration or via other parenteral routes, i.e., intradermally, intranasally, or intravenously.

Generally, the vaccine will usually be presented as a pharmaceutical formulation including a carrier or excipient, for example an injectable carrier such as saline or a pyrogenic water. The formulation may be prepared by conventional means. It will be understood, however, that the specific dose level for any particular recipient animal will depend upon a variety of factors including age, general health, and sex; the time of administration; the route of administration; synergistic effects with any other drugs being administered; and the degree of protection being sought. Of course, the administration can be repeated at suitable intervals if necessary.

The present invention further provides a vaccine formulation comprising nucleic acid and/or polypeptide sequences as described herein or identified by the methods described herein. Optionally the formulation may further comprise a suitable carrier therefore.

The present invention will now be described by way of example and figures as follows:

FIG. 1. Number of mice in each vaccinated group exhibiting mycoplasmaemia after intraperitoneal challenge

FIG. 2. Anti-Mycoplasma immune responses (measured by ELISA) in BALB/c mice before and after vaccination and pre and post challenge. Error bars are standard deviations of the mean OD_{492 nm} for each mouse on each separate bleed. Key: [symbol] pre-vaccination, [symbol] post-vaccination, [symbol] post-challenge. Group 1: Lambda A8; Group 2: Lambda B1; Group 3: Lambda gt11.

FIG. 3. Anti-mycoplasma (dead and alive) immune cell proliferative responses (measured by LSA) in BALB/c mice after challenge with whole Mycoplasma. Key: [symbol] Media (−ve control), [symbol] Live Mycoplasma, [symbol] Dead Mycoplasma.

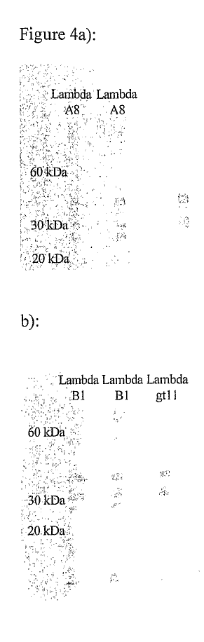

FIG. 4 a) Immunoblot of mice 4 and 8 from group 1 (λA8) and 3 (λgt11) against A8 proteins. Rabbit anti-serum used for positive control. b) Immunoblot of mice 4 and 8 from group 2 (λB1) and 3 (λt11) against B1 proteins. Rabbit anti-serum used for positive control.

FIG. 5. Schematic diagram outlining basic principle of phage DNA vaccination. A strong immune response to the “vaccine” proteins is seen circa 4 weeks post vaccination.

FIG. 6. A8 clone nucleotide and amino acid sequences. A8 nucleotide sequence—7066 base pairs. From 449 864-456 930 in the published genome sequence.
a) Sequence data from the t3 primer of A8 clone (Italics=vector sequence);
b) Sequence data from the t7 primer of A8 clone (Italics=vector sequence)
c) Full A8 nucleotide sequence taken from the published sequence of Mycoplasma mycoides subsp. mycoides SC;
d) Open reading frames of A8 clone;
(i) CDS 449911.450624; codon_start=1; evidence=Not experimental; transl_table=4; Locus_tag=“MSC_{—}0397”; gene=“lpp”; product=“Prolipoprotein”.
(ii) CDS 450876.452816; codon_start=1; evidence=Not Experimental; translatable=4; locus_tag=“MSC_{—}0398”; gene=“natA”; product=“Na+ABC transporter, ATP-binding component”.
(iii) CDS 452976.453563; codon_start=1; evidence=Not_experimental; transl_table=4; locus_tag=“MSC_{—}0399”; product=“hypothetical transmembrane protein”.
(iv) CDS 453718.454629; codon_start=1; evidence Not_experimental; transl_table=4; locus_tag=“MSC_{—}0400”; product=“hypothetical transmembrane protein”.
(v) CDS 454681.454815; codon_start=1; evidence=Not_experimental; transl_table=4; locus_tag=“MSC_{—}0401”; gene=“lpp”; product=“Prolipoprotein”.
(vi) CDS 455051.457189; codon_start=1; evidence=Not_experimental; transl_table=4; locus_tag=“MSC_{—}0402”; product=“Conserved hypothetical protein”.

FIG. 7. B1 clone nucleotide and amino acid sequences
a) B1 clone nucleotide sequence (From 304 656-311 346 in the published genome sequence).
b) Sequence data from the t3 primer of B1 clone (Italics=vector sequence)
c) Sequence data from the t7 primer of B1 clone (Italics=vector sequence)
d) Open reading frames of B1 clone.
(i) CDS 304413.305402; codon_start=1; evidence=Not_experimental; transl_table=4; locus tag=“MSC_{—}0266”; gene=“pdhB”; product=“Pyruvate dehydrogenase (lipoamide), beta chain”; EC_number=“1.2.4.1”
(ii) CDS 305431.306717; codon_start=1; evidence=Not_experimental; transl_table=4; locus tag=“MSC_{—}0267”; gene=“pdhC”; product=“dihydrolipoamide S-acetyltransferase”; EC_number=“2.3.1.12”.
(iii) CDS 306736.308523; codon_start=1; evidence=Not_experimental; transl_table=4; locus_tag=“MSC_{—}0268”; gene=“pdhD”; product=“dihydrolipoamide dehydrogenase”; EC_number=“1.8.1.4”.
(iv) CDS 308545.309513; codon_start=1; evidence=Not_experimental; transl_table=4; locus tag=“MSC_{—}0269”; gene=“pta”; product=“phosphate acetyltransferase”; EC_number=“2.3.1.81”.
(v) CDS 309526.310707; codon_start=1; evidence=Not_experimental; transl_table=4; locus_tag=“MSC_{—}0270”; gene=“ackA”; product=“acetate kinase”; EC_number=“2.7.2.1”.
(vi) CDS 310777.312732; codon start=1; evidence=Not_experimental; transl_table=4; locus_tag=“MSC_{—}0271”; gene=“lpp”; product=“Prolipoprotein”.

### Materials and Methods

### Mycoplasma Strains and Growth Conditions

MmmSC challenge strain N6 (March et al., 2000a) was obtained from Willie Amanfu, National Veterinary Laboratory, Gaborone, Botswana. Mycoplasmas were grown in Gourlay's broth or agar (1%) medium (modified Newings tryptose broth; Gourlay, 1964), containing thallous acetate 0.04% and ampicillin 0.4 mg/ml unless otherwise stated, at 37° C., in an atmosphere containing CO₂ 5%. For the challenge experiment, mycoplasmas were grown to mid-logarithmic phase, concentrated 10-fold by centrifugation and re-suspended in fresh medium immediately before challenge to give a titre of 10¹⁰ organisms/ml.

### Rabbit Antisera

Rabbit hyperimmune serum (R55) against MmmSC strain N6 was produced by two subcutaneous injections of mycoplasma (gluteraldehyde inactivated, followed by quenching with glycine). Sterility was confirmed by the absence of growth after streaking on Gourlay's agar plates. The final vaccine constitution was 5.6 mg/ml protein in phosphate-buffered saline (PBS) containing gluteraldehyde 0.01%, 0.01M glycine, thimerosal 0.01% in an equal volume of oil adjuvant (Montanide ISA50; Seppic, 75, quai d'Orsay, 75321 Paris, France), followed by one intravenous injection of an aqueous suspension. Injections were given every three weeks.

### Preparation of λ-A8 and λ-B1 Clones

DNA prepared by phenol/chloroform extraction from vaccine strain T1/44
Ethanol precipitated and run on agarose gel to check yield and purity
Digested with Tsp509 I
MmmSC genomic DNA was cloned into the unique EcoR I site of the expression vector λ ZAP Express, in which the inserts are under the control of both prokaryotic and eukaryotic promoters.
Resulting library amplified (recombinant phage was plated on E. coli) and the plaques were:
   probed with specific labelled insertion sequences IS1296 (Cheng, 1995) and IS1634 (Vilei, 1999) by southern blotting
   screened (western blot) using IgG purified from hyperimmune serum from a rabbit inoculated with MmmSC strain N6 (March, 2000b).
   Positive clones were picked, plated and re-probed to confirm positive reaction
   Positive clones probed with bovine convalescent sera from 3 diff cattle (pre-adsorbed with E. coli cells lysed with a λ-ZAP express vector without any insert, to reduce non-specific background) to identify those expressing proteins which may elicit an immune response during infection
   The phagemids excised from two strongly immunodominant clones have been named λ-A8 and λ-B1.

### Sequence Using Internal Priming Sites

Express recombinant proteins in E. coli and check sizes using immunoblotting and rabbit anti-serum (Makrina's blot and gel), about 30 and 65 Kb
Grow clones up in E. coli
A8 is IPTG inducible (expression controlled by the promoters β-gal and CMV)

### Growth of Bacteriophage λ

The commonly used cloning vehicle k-gt11 was used throughout. A single colony of E. coli strain Y1090 was added to 100 ml NZCYM broth (Fluka, Biochemika, Switzerland) and grown overnight at 37° C., with vigorous agitation. The cell concentration was calculated, assuming 1OD₆₀₀=8×10⁸ cells/ml. Four aliquots of the E. coli suspension, each containing 10¹⁰ cells were centrifuged at 3300 g for 5 minutes at room temperature (19° C.). Each bacterial pellet was re-suspended in 3 ml phage (SM) buffer (5.8% Sodium Chloride (Fisher Scientific, UK Ltd.), 2% Magnesium sulphate (Fisher Scientific, UK Ltd.), 50 mM Tris.HCl (Sigma Ltd. UK), 2% Gelatine (Sigma Ltd. UK)). 1 μl of λ-gt11/λ-HBsAg bacteriophage was added to each bacterial suspension and incubated at 37° C., with intermittent shaking for 20 minutes. Each infected aliquot was then added to pre-warmed (37°) flasks of 500 ml NZCYM broth and left at 37° C., with vigorous agitation overnight. 10 ml of chloroform (Fisher Scientific, UK Ltd.) was added to each of the flasks, which were then incubated for 10 minutes at 37° C., with shaking. The flasks were left to stand at an angle to allow the chloroform to pool to the bottom, before being removed with a pipette.

Purification of λgt11-Bacteriophage

Lysed cultures were cooled to room temperature. Deoxyribonuclease I (Sigma Ltd. UK) and Ribonuclease A (Sigma Ltd. UK) were added to each flask at a final concentration of 1 μg/ml in each. These were incubated for 30 minutes at room temperature before the addition of 1M Sodium Chloride, which was dissolved before the flasks were left to stand on ice for an hour. Cell debris was removed by centrifugation at 11,000 g for 10 minutes at 4° C. and the collected supernatants were pooled. Solid Polyethylene glycol (PEG, Sigma Ltd. UK) was added to a final concentration of 10% (w/v), dissolved slowly at room temperature, then the flasks incubated at 4° C. for at least one hour. The precipitated bacteriophage particles were recovered by centrifugation at 11,000 g for 10 minutes at 4° C. and the pellet re-suspended in SM buffer (8 ml for each 500 ml of supernatant). The PEG was removed by the addition of an equal volume of chloroform, before centrifugation at 300g for 15 minutes at 4° C. The aqueous phase was then recovered and ultra-centrifuged at 26,000 rpm for 2 hours at 4° C. The bacteriophage pellet was re-suspended in 1-2 ml of SM buffer and incubated at 4° C. overnight.

Titration of λgt11-Bacteriophage

E. coli was diluted 1/10 in Luria broth (Sigma Ltd. UK), (containing 10 mM Magnesium sulphate and 0.2% Maltose (Sigma Ltd. UK)) and incubated overnight at 37° C., with vigorous shaking. This overnight culture was sub-cultured 1/10 in Luria broth (supplemented with 10 mM Magnesium sulphate and 0.2% Maltose) and incubated at 37° C., with vigorous shaking, for 3 hours. The culture was then centrifuged at 3300 g for 5 minutes at room temperature and the pellet re-suspended in 10 mM Magnesium sulphate to an OD₆₀₀ of 0.6.

Appropriate serial dilutions of the purified λ-gt11 bacteriophage suspension were prepared in SM buffer. 5 μl of each dilution was diluted in 195 μl of the E. coli culture and incubated at 37° C. for 15 minutes.
Each bacteriophage/E. coli mixture was added to 2.5-3 ml NZCYM top agar (0.6% purified agar (Oxoid Ltd. England), mixed gently and poured onto Luria broth agar (1% purified agar) plates. The plates were left to set at room temperature for 5 minutes, then incubated at 37° C. overnight. The plaques present on each plate were counted and the λ-gt11 bacteriophage concentration per ml calculated.

### Immunisation of Mice

Mice were strain BALB/c, female and 10 weeks of age, eight per group. All mice were injected intramuscularly on week 0, 4 and 8 with 50 μl SM saline buffer containing 5×10⁸ bacteriophage particles. The 3 groups used were as follows: (1) λ-A8; (2) λ-B1 and (3) negative control, λgt11. Mice were tail bled pre-vaccination at week 0, and post vaccination at weeks 4 and 8, then again on days 0, 2 and 3 post-challenge at week 13. Finally, all mice were humanely killed and bled out on day 4 post-challenge and spleens obtained for stimulation assays.

### Challenge Experiment and Mycoplasmaemia Detection

At week 13, all three groups of BALB/c mice (8 mice per group) were challenged by the intraperitoneal injection of 0.5 ml (10¹⁰ organisms) of MmmSC strain N6, grown in Gourlay's broth without thallous acetate or ampicillin. (Previous research had indicated that this strain produced a particularly high degree of mycoplasmaemia in mice [March and Brodlie 2000]. All mice were held in negative pressure isolators (one group per isolator) to satisfy current disease security requirements. A drop of blood from tail-tip bleeds taken on days 2, 3 and 4 after challenge, was placed in 3 ml of liquid growth medium before being diluted 1 in 10 in the same medium and incubated at 37° C. for 7 days. Growth in liquid medium (observed as a colour change from red to yellow of the 1 in 10 dilutions) was confirmed with an MmmSC-specific latex agglutination test (March et al. 2000b; March and Cloughley 2001), or by plating on solid medium and observing mycoplasma colonies.

### ELISA Analysis of Sera

Antibody titres against bacteriophage λ coat proteins and whole MmmSC antigens in mouse sera (pre-vaccination, pre-challenge and final bleed post-challenge) were measured by indirect ELISA. Microtitre plates (96-well; Greiner Ltd, Brunel Way, Stonehouse, Gloucestershire) were coated overnight at 4° C. in 0.05M sodium carbonate buffer at pH 9.2 with either whole sonicated MmmSC(N6) at a concentration of 5 μg/ml or 10⁹ bacteriophage (50 ng) per well. Plates were blocked for 30 minutes with PBS supplemented with Tween 20 (Sigma) 0.5% (PBST) and dry skimmed milk 5% w/v (blocking buffer) before incubation with a 1 in 400 dilution of primary mouse antiserum in blocking buffer (100 μl/well) overnight at 4° C., in triplicate. Plates were subsequently washed three times with PBST and incubated with goat anti-mouse horseradish peroxidase [HRP]-labelled secondary antibody (DAKO A/S, Glostrup, Denmark) diluted 1 in 2000 in blocking buffer for 2 hours at 37° C. Plates were then washed three times in PBST and “developed” with o-phenylenediamine dichloride (OPD) (Sigma), in the dark at room temperature for 10 minutes. The reaction was stopped by the addition of 3M H₂SO₄ and the optical density (OD) was measured at 492 nm with an IEMS Plate Reader (Labsystems, Life Sciences International, Edison Road, Basingstoke, Hampshire).

### Lymphocyte Stimulation Assay (LSA)

At week 13, day 4 following challenge, the spleens were harvested from each mouse and combined for each of the four groups (8 spleens per group). The splenocytes were recovered from the spleens via injection with 2 ml mouse wash medium (MWM, containing Hanks Balanced salt Solution, 2% Foetal Bovine Serum (FBS), 2% Penicillin/Streptomycin, 0.25% Nystatin and 0.2% gentamycin (Lloyds Chemist PLC., UK)) and light bashing with sterile microscope slides. This cell suspension was then filtered through lens tissue (Whatman International Ltd, England), before being centrifuged for 5 minutes at 1500 rpm at 16° C. The pellet was re-suspended in lysis buffer (nine parts 0.16M ammonium chloride (Sigma Ltd., UK) and one part 0.17M Tris (Sigma Ltd., UK) at pH 7.65). After 10 minutes at 4° C., lysis was stopped by the addition of MWM. Following centrifugation at 1500 rpm for 5 minutes at 16° C., the pellet was re-suspended in complete RPMI (RPMI 1640, 10% FBS, 2% glutamine, 1% penicillin/streptomycin, 1% gentamycin, 0.5% 2-mercaptoethanol, 2.5% Sodium Bicarbonate (8%) and 1.2% 1M Hepes (Sigma Ltd, UK)). This cell suspension was centrifuged again (as above), and the pellet re-suspended in 1 ml complete RPMI. A 1:10 dilution of cell suspension in 0.1% nigrosin (Sigma Ltd., UK) in phosphate buffered saline was prepared for a viability count, using a modified Neubauer counting chamber. Cell concentrations were adjusted to 1×10⁶ cells/ml in complete RPMI. Using sterile 96-well tissue culture plates, 100 μl of the viable splenocytes were seeded onto 100 μl volumes of sterile diluted antigens in triplicate. Mycoplasma live and dead (heat killed) were diluted in complete RPMI, to give a concentration of 10⁶ cells/ml for each. Also used was λgt11, diluted in complete RPMI to give concentrations of 5 and 2.5 μg/ml. As a positive control, cells were cultured with concavalin A (Sigma Ltd., UK) at a concentration of 2.5 μg/ml. Plates were incubated in a humid (5% CO2) environment at 37° C. for 96 hours. After 4 days incubation, the cell cultures were pulsed for 18 hours with 18.25 KBq (1 μCi) [3H] thymidine (Amersham Biosciences UK), per well. The cells were harvested in a Packard Harvester onto glass fibre filters (Packard, Netherlands), and activity counted in a direct beta counter (Packard, Netherlands). Results are expressed as average counts per minute (cpm) (±standard deviation), from which stimulation indices (SI) were calculated using the following equation: SI=average cpm of test/average cpm of media control (no antigenic stimulus).

### Statistical Analysis

The immune responses were analysed by applying the Mann-Whitney test to comparisons of the mean weekly increases in the ELISA titre. The results of challenge were analysed by applying the one-sided Fisher's Exact Probability test to comparisons of the numbers of mycoplasmaemic mice in different groups on days 2 and 3.

### Immunoblots

Clones A8 and B1 were prepared in SDS-PAGE sample buffer (0.1M Tris-HCl pH 6.8, 40% (v/v) glycerol, 4% (w/v) SDS, 0.25% bromophenol blue, 2% β-mercaptoethanol). The samples were boiled for 5 minutes and separated by SDS-PAGE using a 12% homogeneous polyacrylamide gel followed by electrophoresis transfer to nitrocellulose membranes (Hybond C-pure, Amersham, UK) using standard techniques. Rainbow markers (Amersham, UK) were run alongside the samples. Efficiency of transfer was estimated by staining the membranes with 0.1% Ponceau Red (Sigma) in 1% acetic acid solution, followed by destain in 1% acetic acid. The membrane was then rinsed twice in PBST (PBS+0.5% Tween 20), and blocked in 5% (w/v) dry skimmed milk in PBST for 30 minutes before primary specific mouse polyclonal antibody was added at a dilution of 1:100. Biorad Mini-Protean II Multi-Screen blotting apparatus was used to allow multiple primary serum samples to be tested against the same protein. The membrane was subsequently incubated for 1 hour at room temperature, rinsed five times in PBST, then anti-mouse horse radish peroxidase-labelled secondary antibody (DAKO) was added diluted in 5% (w/v) dry skimmed milk in PBST as per manufacturers instructions, and the membrane incubated for a further hour at room temperature. Three 5 minute washes in PBST were performed, before incubation of the membrane in 0.1 mg/ml diaminobenzidine (Sigma) in PBST containing 0.1 ml of 30% H₂O₂ per 100 ml of substrate solution.

### Results

### Preparation of λ-A8 and λ-B1 Clones

Examination of the amplified λ-ZAP express library with the specific labelled insertion sequences confirmed that library construction was successful and that a high proportion of the clones contained T1/44 DNA. A significant number of clones were positive, when tested with both rabbit and immune serum and bovine convalescent sera.

### Challenge Experiment and Mycoplasmaemia Detection

Table 1a, b and c show the results of challenge (week 13) with Mycoplasma. Group 1 mice, immunized with the λ-A8 vaccine, were almost completely protected from infection with live mycoplasma, as judged by the absence of mycoplasmaemia (only ⅛ still infected) on day 4 post-challenge. Less animals in group 2 (3 out of 8 mice), vaccinated with the λ-B1 vaccine had successfully cleared the infection from the blood by day 4 as compared with the control group 3 (immunised with λ-gt11), where 4 out of the 8 mice were protected.

### Immune Responses of Mice to Bacteriophage λ Coat Proteins and Whole MmmSC Antigens, as Measured by ELISA

The whole MmmSC immune responses before and after vaccination and following challenge, for the three different mouse groups are shown in FIG. 2. Five out of eight mice in group 1 (vaccinated with λ-A8 construct) show a higher response (OD value) against whole Mycoplasma in the post vaccination sera compared to that of the pre-vaccination sera. This was most apparent in mice 2 and 5, where an OD value of >1.5 is seen. In group 2 mice (vaccinated with λ-B1 construct) only two out of the eight mice gave a higher response in the post-vaccination bleed compared to pre-vaccination bleed. However, in comparison to group 3 mice (vaccinated with λ-gt11), where no mice showed this rise in response to whole Mycoplasma, the OD values are a lot higher. All three groups have an increased response to whole Mycoplasma following challenge when compared to pre-vaccination sera OD values. However, this is in response to whole Mycoplasma, which contains many different proteins and so is not showing a specific response to A8 and B1 proteins themselves.

Immune Responses of Mice to Whole Live/Dead Mycoplasma and λgt11, as Measured by LSA

Following four days culture, cellular proliferation was measured by the incorporation of ³H-thymidine (counts per minute (cpm)), in response to stimuli from whole live/dead mycoplasma and phage (λ-gt11). From these values, the stimulation index for each group, in response to whole mycoplasma, was calculated (FIG. 3). The spleens from each group were combined to give an overall proliferation for each group. Therefore, the standard deviation values are fairly large, due to animal variation within groups. However, a weak response is still seen in groups 1 and 2 following stimulation with live whole mycoplasma, with SI values of 2.5 for both. The responses to phage (results not shown) were high for all 3 groups, giving SI values between 20 and 346, suggesting a phage specific T cell-mediated immune response was induced.

### Immunoblots

Specific immune responses were detected by immuno-blotting mice anti-sera from groups 1, 2 and 3 final bleeds against A8 and B1 proteins. The two mice tested in group 1 (mouse 4 and 8, vaccinated with the λ-A8 construct), against E. coli expressing the A8 protein, each showed a positive band of <30 kD, compared to group 3 serum (mouse 4, vaccinated with λgt11), which showed no positive band of this size (FIG. 3a)). This was also the case for the mice sera from group 2 (mouse 4 and 8, vaccinated with the λ-B1 construct), when tested against E. coli expressing the B1 protein. Sera from both mice showed two distinct positive bands at <30 kD and <60 kD, compared with that of group 3 mouse (mouse 4, vaccinated with λgt11), which had no visible bands of these sizes (FIG. 3b)). Previous results from immunoblots (not shown) of rabbit anti-MmmSC serum against E. coli containing A8 or B1 plasmids showed positive bands of sizes 28 kD and 30 and 60 kD, respectively. This together with these results suggests that antiserum from mice vaccinated with the lambda A8 and B1 constructs recognised size specific recombinant MmmSC proteins expressed in E. coli.

### Discussion

A mouse infection technique (Smith, 1965, 1969, 1971a,b) was used to test the vaccines. A protracted mycoplasmaemia could be produced in laboratory mice by intraperitoneal inoculation with approximately 10¹⁰ viable organisms in growth medium. In some cases (group 1, A8 vaccinated mice), vaccination produced a protective immune response, demonstrated by a reduction in the mycoplasmaemia produced by challenge.

The factors that may affect the mouse immune response to MmmSC antigens have not been evaluated. It has been suggested that C57BL/6 mice are resistant to infection with the human pathogen Mycoplasma pneumoniae because of an innate immunity associated with alveolar macrophages and humoral immunity (Hickman-Davis et al., 1997).

A relation was observed between a high anti-MmmSC titre and protection against challenge, Smith (1971b) showed that mice vaccinated intravenously with heat-killed MmmSC were completely protected from mycoplasmaemia after challenge with live organisms. However, when the serum from these vaccinated mice (0.25 ml) was transferred to unvaccinated mice, they were not protected upon challenge with live organisms; however, a dose of 1 ml of undiluted serum per mouse was slightly protective. In contrast, the same volume of a 1 in 50 dilution of serum taken from rabbits after recovery from infection with MmmSC protected mice from mycoplasmaemia. Thus, the rabbit antiserum was much more protective than the mouse antiserum. When cattle, rabbits and mice were immunized subcutaneously with heat-killed MmmSC in adjuvant, the mouse-protective effective of bovine and rabbit antisera was high; in contrast, however, the protection of mice against challenge was slight or nil (Hooker et al., 1980). These observations are consistent with findings that rabbit serum R54 inhibited MmmSC growth but mouse serum did not.

### REFERENCES

Abusugra, I. and Morein, B. (1999). ISCOM is an efficient mucosal delivery system for Mycoplasma mycoides subsp. mycoides (MmmSC) antigens inducing high mucosal and systemic antibody responses. FEMS Immunology and Medical Microbiology, 23, 5-12.
Abusugra, I., Wolf, G., Bolske, G., Thiaucourt, F. and Morein, B. (1997). ISCOM vaccine against contagious bovine pleuropneumonia (CBPP). 1. Biochemical and immunological characterization. Veterinary Immunology and Immunopathology, 59, 31-48.
Balcer, V. and Dedieu, L. (2000). Cell-mediated immune response induced in cattle by Mycoplasma mycoides subsp. mycoides: comparison between infected and vaccinated animals. In: Mycoplasmas of Ruminants: Pathogenicity, Diagnostics, Epidemiology and Molecular Genetics. COST 826. Vol. 4, D. Bergonnier, X. Berthelot and J. Frey, Eds, European Union, Luxembourg, pp. 97-100.
Bergquist, C., Lagergard, T. and Holmgren, J. (1998). Antibody responses in serum and lung to intranasal immunization with Haemophilus influenzae type b polysaccharide conjugated to cholera toxin B subunit and tetanus toxoid. APMIS, 106, 800-806.
Buttery, S. H., Lloyd, L. C. and Titchen, D. A. (1976). Acute respiratory, circulatory and pathological changes in the calf after intravenous injections of the galactan from Mycoplasma mycoides subsp. mycoides. Journal of Medical Microbiology, 9, 379-391.
Buttery, S. H. and Plackett, P. (1960). A specific polysaccharide from Mycoplasma mycoides. Journal of General Microbiology, 23, 357-368.
Cartner, S. C., Lindsey, J. R., Gibbs-Erwin, J., Cassell, G. H. and Simecka, J. W. (1998). Roles of innate and adaptive immunity in respiratory mycoplasmosis. Infection and Immunity, 66, 3485-3491.
Cheng, X., J. Nicolet, F. Poumarat, Regalla, J., F. Thaicourt, and J. Frey. (1995). Insertion element IS1296 in Mycoplasma mycoides subsp. mycoides small colony identifies a European clonal line distinct from African and Australian strains. Microbiology, 141, 3221-3228.
[Clark J R & March J B. FEMS Immunol. Med. Microbiol. 40 (1), 21-26 (2004). March J B, et al. Vaccine 22 (13-14), 1666-1671 (2004)].
Dubois M., Gilles K., Hamilton J. K., Rebers P. A. and Smith, F. (1956). Colorimetric method for determination of sugars and related substances. Analytical Chemistry, 28, 350-356.
Ey, P. L., Prowse, S. J. and Jenkin, C. R. (1978). Isolation of pure IgG1, IgG2a and IgG2b immunoglobulins from mouse serum using protein A-sepharose. Immunochemistry, 15, 429-436.
Garba, S. A., Ajaya, A., Challa, L. D., Bello, M. K., Gazama, J. N. Z., Gimba, H., Ngbede, J., Bitrus, B. and Adeoye, A. O. (1986). Field trial of inactivated oil-adjuvant Gladysdale strain vaccine for contagious bovine pleuropneumonia. Veterinary Record, 119, 376-377.
Garba, S. A., Terry, R. J., Adegboye, D. S., Lamorde, A. G. and Abalaka, J. A. (1989). The choice of adjuvants in mycoplasma vaccines. Microbios, 57, 15-19.
Gengoux, C. and Leclerc, C. (1995). In vivo induction of CD4+ T cell responses by antigens covalently linked to synthetic microspheres does not require adjuvant. International Immunology, 7, 45-53.
Goebel, W. F. and Avery, O. T. (1929). Chemo-immunological studies on conjugated carbohydrate-proteins I. The synthesis of p-aminophenol β-glucoside, p-aminophenol β-galactoside, and their coupling with serum globulin. Journal of Experimental Medicine, 50, 521-531.
Goldblatt, D. (1998). Recent developments in bacterial conjugate vaccines. Journal of Medical Microbiology, 47, 563-567.
Gourlay, R. N. (1964). Antigenicity of Mycoplasma mycoides. I. Examination of body fluids from cases of contagious bovine pleuropneumonia. Research in Veterinary Science, 5, 473-482.
Gray, M. N., Simam, P. and Smith G. R. (1986). Observations on experimental inactivated vaccines for contagious bovine pleuropneumonia. Journal of Hygiene. London, 97, 305-315.
Hickman-Davis, J. M., Michalek, S. M., Gibbs-Erwin, J. and Lindsey, J. R. (1997). Depletion of alveolar macrophages exacerbates respiratory mycoplasmosis in mycoplasma-resistant C57BL mice but not mycoplasma-susceptible C3H mice. Infection and Immunity, 65, 2278-2282.
Hooker, J. M., Smith G. R. and Milligan, R. A. (1980). Immune response of mice, rabbits and cattle to inactivated Mycoplasma mycoides subspecies mycoides vaccines containing adjuvants. Journal of Comparative Pathology, 90, 363-372.
Howard, C. J., Gourlay, R. N. and Taylor, G. (1980). Immunity to Mycoplasma bovis infections of the respiratory tract of calves. Research in Veterinary Science, 28, 242-249.
Howard, C. J. and Taylor, G. (1985). Immune responses to mycoplasma infections of the respiratory tract. Veterinary Immunology and Immunopathology, 10, 3-32.
Hudson, J. R., Buttery S. H. and Cottew, G. S. (1967). Investigations into the influence of the galactan of Mycoplasma mycoides on experimental infection with that organism. Journal of Pathology and Bacteriology, 94, 257-273.
Hyslop, G. (1958). The adaptation of Asteroccocus mycoides to rodents. Journal of Pathology and Bacteriology, 56, 189-199.
Konadu, E., Robbins, J. B., Shiloach, J., Bryla, D. A. and Szu, S. C. (1994). Preparation, characterization, and immunological properties in mice of Escherichia coli 0157 0-specific polysaccharide-protein conjugate vaccines. Infection and Immunity, 62, 5048-5054.
Krieg, A. M., (1996) Trends in Microbiology, 4, 73-77.
[Leitner W W, et al. Vaccine 18 (9-10), 765-77 (1999)],
Lloyd, L. C., Buttery, S. H. and Hudson, J. R. (1971). The effect of the galactan and other antigens of Mycoplasma mycoides var. mycoides on experimental infection with that organism in cattle. Journal of Medical Microbiology, 4, 425-439.
Loiselet, R., Ball, H. J., and March, J. B. (2001). Analysis of capsular polysaccharide purified from Mycoplasma mycoides subsp. mycoides small colony with monoclonal antibodies. In: Mycoplasmas of Ruminants, Pathogenicity, Diagnostics, Epidemiology and Molecular Genetics. COST 826. Vol. 5, J. B. Poveda, A. S. Fernandez., J. Frey and K.-E. Johansson, Eds, European Union, Luxembourg, pp. 64-67.
March, J. B. and Brodlie, M. (2000a). Comparison of the virulence of European and African isolates of Mycoplasma mycoides subspecies mycoides small colony type. Veterinary Record, 147, 20-21.
March, J. B., Clark, J. and Brodlie, M. (2000b). Characterization of strains of Mycoplasma mycoides subsp. mycoides small colony type isolated from recent outbreaks of contagious bovine pleuropneumonia in Botswana and Tanzania: evidence for a new biotype. Journal of Clinical Microbiology, 38, 1419-1425.
March, J. B. and Cloughley, C. (2001). Detection of contagious bovine pleuropneumonia using a capsular polysaccharide-specific latex agglutination test. American Society for Microbiology 101st General Meeting, Florida, May 20-24, 2001, p. 391.
March, J. B., Gammack, C. and Nicholas, R. (2000). Rapid detection of contagious caprine pleuropneumonia using a Mycoplasma capricolumn subsp. capripneumoniae capsular polysaccharide-specific antigen detection latex agglutination test. Journal of Clinical Microbiology, 38, 4152-4159.
March, J. B. and Jones, G. E. (1998). Inhibitory effects of vaccines containing subunit fractions of Mycoplasma capricolumn subsp. capripneumoniae. In: Mycoplasmas of Ruminants: Pathogenicity, Diagnostics, Epidemiology and Molecular Genetics. COST 826, Vol. 2, G. Leori, F. Santini, E. Scanziani and J. Frey, Eds, European Union, Luxembourg, pp. 44-49.
March, J. B., Jones, G. E., Hitchen, P., Morris, H. R. and Dell, A. (1999). Analysis of the capsular polysaccharide of Mycoplasma mycoides subsp. mycoides SC, the causal agent of CBPP: purification, composition and its role in infection and immunity. In Mycoplasmas of Ruminants: Pathogenicity, Diagnostics, Epidemiology and Molecular Genetics. COST 826, Vol. 3, L. Stipkovits, R. Rosengarten and J. Frey, Eds, European Union, Luxembourg, pp. 69-72.
Markham, J. F., Morrow, C. J. and Whithear, K. G. (1998). Efficacy of a temperature-sensitive Mycoplasma synoviae live vaccine. Avian Diseases, 42, 671-676.
Masiga, W. N., Roberts, D. H., Kakoma, I. and Rurangirwa, F. R. (1975). Passive immunity to contagious bovine pleuropneumonia. Research in Veterinary Science, 19, 330-332.
Masiga, W. N. and Windsor, R. S. (1975). Immunity to contagious bovine pleuropneumonia. Veterinary Record, 97, 350-351.
Mu, H. H., Sawitzke, A. D. and Cole, B. C. (2000). Modulation of cytokine profiles by the mycoplasma superantigen Mycoplasma arthritidis mitogen parallels susceptibility to arthritis induced by M. arthritidis. Infection and Immunity, 68, 1142-1149.
Mulira, G. L., Masiga, W. N. and Nandokha, E. (1988). Efficiency of different adjuvants to potentiate the immune response to mycoplasma strain F-3 8. Tropical Animal Health and Production, 20, 30-34.
O.I.E. (1996). Contagious bovine pleuropneumonia. In: Manual of standards for Diagnostic Techniques and Vaccines, Office International des Epizooties, Paris, pp. 85-92.
Okada, M., Sakano, T., Senna, K., Maruyama, T., Murofushi, J., Okonogi, H. and Sato, S. (1999). Evaluation of Mycoplasma hyopneumoniae inactivated vaccine in pigs under field conditions. Journal of Veterinary Medical Science, 61, 1131-1135.
Plackett, P. and Buttery, S. H. (1964). A galactofuranose dissacharide from the galactan of Mycoplasma mycoides. Biochemical Journal, 90, 201-205.
Rurangirwa, F. R., Masiga, W. N. and Muthomi, E. K. (1984). Immunisation of goats against contagious caprine pleuropneumonia using sonicated antigens of F-38 strain of mycoplasma. Research in Veterinary Science, 36, 174-176.
Rurangirwa, F. R., McGuire, T. C., Kibor A. and Chema, S. (1987a). An inactivated vaccine for contagious caprine pleuropneumonia. Veterinary Record, 121, 397-400.
Rurangirwa, F. R., McGuire, T. C., Magnuson, N. S., Kibor, A. and Chema, S. (1987b). Composition of a polysaccharide from mycoplasma (F-38) recognised by antibodies from goats with contagious pleuropneumonia. Research in Veterinary Science, 42, 175-178.
Rweyemamu, M. M. (2001). Overview of the objectives of the consultation and expected outputs-CBPP vaccines. In: Report of the Second Meeting of the FAO/OIE/OAU-IBAR Consultative Group on Contagious Bovine Pleuropneumonia (CBPP), Rome, 24-26 Oct. 2000, FAO publication X9110/E, Food and Agriculture Organisation, Rome, Italy, pp. 3-9.
Rweyemamu, M. M., Litamoi, J., Palya, V. and Sylla, D. (1995). Contagious bovine pleuropneumonia vaccines: the need for improvements. Revue Scientifique et Technique, Office International Des Epizooties, 14, 593-601.
Saito, S., Fujisawa, A., Ohkawa, S., Nishimura, N., Abe, T., Kodama, K., Kamogawa, K., Aoyama, S., Iritani, Y. and Hayashi, Y. (1993). Cloning and DNA sequence of a 29 kilodalton polypeptide gene of Mycoplasma gallisepticum as a possible protective antigen. Vaccine, 11, 1061-1066.
Sambrook, J. and Russel, D, Molecular Cloning: A laboratory manual, 2001, Cold Spring Harbor Laboratory Press.
Sato, Y. et al (1996) Science, 273, 352-354
Sheriff, D. and Piercy, S. E. (1952). Experiments with an avianised strain of the organism of contagious bovine pleuropneumonia. Veterinary Record, 62, 615-621.
Shibata, Y., Metzger, W. J. and Myrvik, Q. N. (1997). Chitin particle-induced cell-mediated immunity is inhibited by soluble mannan: mannose receptor-mediated phagocytosis initiates IL-12 production. Journal of Immunology, 159, 2462-2467.
Shifrine, M. and Beech, J. (1968). Preliminary studies on living culture and inactivated vaccines against contagious bovine pleuropneumonia. Bulletin of Epizootic Diseases of Africa, 16, 47-52.
Smith, G. R. (1965). Infection of small laboratory animals with Mycoplasma mycoides var. capri and Mycoplasma mycoides var. mycoides. Veterinary Record, 77, 1527-1528.
Smith, G. R. (1969). Effect of route of vaccination on the immune response of mice to a single dose of heat-killed Mycoplasma mycoides var. mycoides. Journal of Comparative Pathology, 79, 255-260.
Smith, G. R. (1971a). The use of mice in research on contagious bovine pleuropneumonia. Tropical Animal Health and Production, 3, 169-172.
Smith G. R. (1971b). Mycoplasma mycoides var. mycoides: immunity and mouse-protective antibody. Journal of Comparative Pathology, 81, 267-278.
Thiaucourt, F., Yaya, A., Wesonga, H., Huebschle, O. J., Tulasne, J. J. and Provost, A. (2000). Contagious bovine pleuropneumonia. A reassessment of the efficacy of vaccines used in Africa. Annals of the New York Academy of Sciences, 916, 71-80.
Vilei, E. M., J. Nicolet, and J. Frey. (1999). IS1634, a novel insertion element creating long, variable-length direct repeats which is specific for Mycoplasma mycoides subsp. mycoides small colony type. J. Bact, 181, 1319-1323.
Waite, E. M., Loiselet, R., and March, J. B. (2000). The use of undefined medium components in the growth of Mycoplasma mycoides subsp. mycoides small colony variant (MmmSC): implications for vaccine production and extraction of the capsular polysaccharide. Program and Abstracts of the 13^{th} International Congress of the International Organization for Mycoplasmology, Fukuoka, Japan, Jul. 14-19, 2000, p. 209.
Waite, E. and March, J. B. (2001). Capsular polysaccharide of Mycoplasma mycoides subsp. mycoides small colony variant: compositional and structural studies. In: Mycoplasmas of Ruminants, Pathogenicity, Diagnostics, Epidemiology and Molecular Genetics. Vol. 5. COST 826. J. B. Poveda, A. S. Fernandez, J. Frey and K.-E. Johansson, Eds, European Union, Luxembourg, pp. 69-72.

1. A method for identifying a nucleic acid sequence or polypeptide for potential use in a vaccine, said method comprising:
a) providing a genetically modified phage comprising nucleic acid from a disease causing agent or diseased cell;
b) expressing a polypeptide(s) encoded by said nucleic acid sequence;
c) contacting said expressed polypeptide(s) with antiserum from an animal which has previously been infected with said disease causing agent and/or has said diseased cell(s); and
d) identifying said polypeptide(s) which specifically reacts with said antiserum.

2. The method according to claim 1 wherein the phage has been modified so as to be capable of expressing the polypeptide(s) in a cell to be infected by said phage and/or to express the polypeptide(s) on the surface of the phage particle, by the technique known as phage display.

3. The method according to claim 1 wherein the polypeptide(s) is expressed in a host cell transfected with said phage.

4. The method according to claim 3 wherein the host cell is Escherichia coli.

5. The method according to claim 1 wherein the phage to be modified by introduction of said nucleic acid from a disease causing agent or diseased cells is λ-gt11 or λZAP express.

6. The method according to claim 1 wherein the antiserum has been obtained from a mammal such as human, cow, sheep, pig, goat, rabbit, mouse or rat.

7. The method according to claim 1 wherein the antiserum has been obtained from a bird, such as a chicken, duck or turkey or other farmed bird, or a fish, such as salmon, sea bass, trout or other farmed fish.

8. The method according to claim 1 wherein the antiserum is brought into contact with said expressed polypeptides by first immobilising the phage to a substrate, such as nitrocellulase and lysing, if appropriate, the phage, in order to allow the polypeptide(s) to be exposed such that they are capable of coming into contact with said antiserum.

9. The method according to claim 1 wherein polypeptides which specifically react with antibodies present in the antiserum are detected by Western blotting or immunoassay.

10. The method according to claim 1 wherein the phage encoding the identified polypeptide is isolated and the polypeptide encoding nucleic acid sequenced in order to ascertain the identity of the polypeptide and/or nucleic acid.

11. A method of prophylactically or therapeutically vaccinating an animal comprising administering to an animal a polypeptide(s) or phage encoding said polypeptide(s) identified by the method according to claim 1.

12. The method according to claim 11 wherein the phage comprises appropriate transcription/translation regulators for controlling expressing of said polypeptide in a eukaryotic cell.

13. A vaccine formulation comprising a polypeptide(s) or phage encoding said polypeptide(s) identified by the method according to claim 1.

14. A modified phage expressing peptides of the pathogen Mycoplasma, e.g., Mycoplasma mycoides subsp. mycoides small colony type (MmmSC) for use as a vaccine to treat or prevent Contagious bovine pleuropneumonia (CBPP).

15. A modified phage(s) according to claim 14, which is capable of expressing a polypeptide encoded by nucleotide sequence A8 as shown in FIG. 6c, or functional fragment, homologue or derivative thereof for use in a vaccine for the prevention of disease caused by the pathogen MmmSC.

16. A modified phage(s) according to claim 14, which is capable of expressing a polypeptide encoded by a nucleotide sequence B1 as shown in FIG. 7a, or functional fragment, homologue or derivative thereof for use in a vaccine for the prevention of disease caused by the pathogen MmmSC.

17. A modified phage(s) according to claim 14, which is capable of expressing a polypeptide or functional fragment, homologue or derivative thereof for use in a vaccine for the prevention of disease caused by the pathogen MmmSC, wherein said polypeptide is at least one prolipoprotein as shown in FIGS. 6(d) or 7(d) or functional fragment, homologue or derivative thereof.

18. The modified phage according to any claim 14 identified by the method according to claim 1.

19. (canceled)

20. A method of prophylactically treating CBPP using a polypeptide, prolipoprotein, lipoprotein and/or nucleic acid encoding a polypeptide, prolipoprotein or lipoprotein as shown in FIGS. 6d and/or 7d or functional fragment, homologue or derivative thereof, the method comprising administering to an animal an effective amount of said polypeptide, prolipoprotein or lipoprotein and/or nucleic acid encoding a polypeptide, prolipoprotein or lipoprotein such that a suitable immunogenic response to said polypeptide, prolipoprotein or lipoprotein is raised in said animal.


SEQUENCE LISTING (18 sequences)

SEQ ID NO: 1 - DNA, 1091 bp, Artificial
  Lambda-A8 clone sequence
ttttaatctnatgccctttgcacccccanncgactttagnngatcccccgaattnttgatgatattnanattctattagcctatgannggatttaaatgaanaagntattaacagnnttnggggntattggtttaattgctacaagtggagttgcngttgttgcatgtaatacatctgataaaacaaaaatgcctaatgaaaataagggtgangaaaaagntgatttaataaaagttgctaagcaaaaagatttaggttttattagtaaaaaagacaatgaaataattaaaaaagcttttattaagcaaaattctattgacgaaaaaaaagtnactgtaagtgttaaaggtaatggaaatggtgtttctgctactggttcagatactaacacaactacaaatacangtaatggtaatttaaaccgatngngctgttattgaattaaaagctacaaataatggnaatgaaacaaaaacagtaactgtaattttgcagtaaataataattttanagaacttaattcangttaaaaaantaaaaggtttagcagataataaagatgatacaattctaaaagcaattgatnaactaaatcctaaatcaaatctaanatacttctanattatcaatttgaaagaaacgataaaaaagtttctntaaagtcatctgatngnacttatacagggaaatcctgttgaaattganattgaatcaaaagtaggagttnntgnnngtctttctcttccttcngtnctttattagctcctcacggattanttnttntttgngnttttnanangnaaacggnattcnnnntttagacttaacngcnnntanaaantccccnttttantcntttttntanaaacannaaaaaaattaaaaatntttttttttnaaaaanccccttnttgggcntgncttttcctctnnnccctttaaanaanttntctnnggccctncntngnncnngnntnttttttttttttttcttgcaccntaanctctngnttncccnnccccnttctncccctgacttnntncctccgntttcntgctnnnttcctcttttttcttttnannnantncn

SEQ ID NO: 2 - DNA, 1096 bp, Artificial
  Lambda-A8 clone sequence
ttaaaatttanantcttacatgccncttntgaaaanccncggtaccntttggcgnaaggaacccccgatggggcccgcggccgctctagaagtactctcgagaagcttttttaatttactttgctcatagtctnngggnnggctttcatttttataataaatntcttgttcatatcgatgttgttgttcttgatcttgaaaaactttaaaatttttaattcctaacatattagaaatcaagtattcttctttaacttttttaacatcagcattaaaatctaagctataaaaactagttcaatcatctactctattattttcagtttgacttaaatatttttctcattcatctcaaatattaaaatcactattttcactatcttttaaactgcttggtttacctgtcatattaaacaatagtctttttagtggatagctaggttctgaactttcatagtctgggtatgccttttgaattccataaaacttatggaattgatctagcattacttcaattcctttataatatgcatcaaatacagcagactttttataaccttcacttgttaaattttctaaattttcaaaattcaactctttatcttttttatcaagttctacaaaataactaaacgaaatattacctaaatcatgtacatatccatttctagttttactgtaagccttttagatcaaaactttcaatagaagtagatgatttaaataaattttttaaacattatctttttctaatttattattaaactcatctaggttaatcttcatcaaccaaatccncttgcaatattagctaatgnttggttaaaacttagttttataattaagncnttttttntcccaaggggttaacnaggcttttggngtaatttctcnactcccggttaagggggaaacaaggttattttnttgcccttctttnnttcnccnaatttanagcacccagncncttgaccttttttaaggacnggtntttacccttgggatattantancncnttttaangaccaaaatnagncggtttatttttttcnnccnancntgnttntgtntttaantaaanaaaaaaannatnct

SEQ ID NO: 3 - DNA, 7066 bp, Mycoplasma mycoides
aatttttgatgatattaatattctattagcctatgaaaggatagaaatgaagaagttattaacaatattaggttctattggtttaattgctacaagtggagttgcagttgttgcatgtaatacatctgataaaacaaaaatgcctaatgaaaataagggtgaagaaaaagttgatttaataaaagttgctaagcaaaaagatttaggttttattagtaaaaaagacaatgaaataattaaaaaagcttttattaagcaaaattctattgacgaaaaaaaagtaactgtaagtgttaaaggtaatggaaatggtgtttctgctactggttcagataataacacaactacaaatacaagtaatggtaatttaaacgatagtgctgttattgaattaaaagctacaaataatggtaatgaaacaaaaacagtaactgtaatttttgaagtaaataataatttagagaaattaattcaagtaaaaaaattaaaaggtttagcagataataaagatgatacaattttaaaagcaattgataaactaaatcctaaatcaaatttagatacttctaaattatcaattgaaagaaaagataaaaaagtttctataaagtcatctgatagtacttatacaggaaatcctgttgaaattgaaattgaatcaaaagtaggagtttatgttggtctttctctactttcagtagctttattagcttcttcaggatttattatttatagaagtttaaaaaagaaaaagaaataaaatattaagactaaaaggcgtataaaaatatgccttttttaattttttttaataaaaaaataaaaaaaatttaaaaattcttttcttataataaagcctattctataggctttttattatacttatcaacattaaagaacttgttaacgagcttttaaaggcttgttttttttttttttttttgtaaaaataaaaataagatttgaagcaaccaagaatacaaaacataaactaaataaaatgtagtctagttgggtttcaaatctgcatatattagtacaaaggaagatatggtaggaaatgtttttgaaaaactacgtgctaatgtgaaaatgaaaagtgataaatatagtatagtagttgataatttttataaaaagtttaaagatatagaaattggtccgttttcatttaatattgaaaaagggaaaattactgctttattaggaagtagtggatctggtaaaagtgtatttattaactcactactaggaacaagtattaattatcaaggaaacatttttattaatcaaaaagaaagaaaagatagcgattcaatccaaaataattctgatattggattttattcacaaatggacttttctttgtactcaatctcagcttatgattttttatataatatgtgctatgttatgggtttagaacaaaaactagttaaaaccagactagaatattgattgaaaaaatttgatttatgagaatataaagataaacctttaaaaagcttttcatgagggatgaaaaatcgtgttaacttaattttatgttttataaaagaacctagaattttagtatgcgatgaacctggagctagcttagattctcattgaagaaatcaaatttataaaatcttagatgaatttagaagaaaatcaaatagtacaataattttaactgttcataatataaatgaagtttatgatattatagaaaactttgtaattttagaaaaaggtaaattactattttgtggtactaaacaagaactaaatttgtataaaaaaacaaagattacattcgaaaataatatattactagatgaagtagaaaaaattttaaatcaaaatgatattttaacttttaatctagatagtgattctaattctttagtaattggtttaaaagaacatcaagtttttagtgatgctttaaatattttagaaaaaaacaattttcaaataaaaagtattgctagtttatctattaatattgatgcaattaaaaaagctttagaagataagatcactaaacatcaacctaaatatcaaccagatattaatttaaactctaattttaaccaatcaattagttttgataaaactttaaataattcaaatgtaaccaatcagcgtgattcagcttttgaacaattagaagttgagatattagataataacaataataataacaataataataacaataataataacaataataataacaataataataacaataataataacaataataataacaataataatattcaatcagttaatttaattaataatttattgcaaaaaacatttttaaatatgtttaatgaaatcaataatttaaaacaacaagttaatcaaagcaatctagataactatgaattttttgtagatgaactaaaagctcaaattaatagtgttagtgaaattaaaaaagattttttagacaaaatagatcaactaaatactgatcaaagtagtgttttagatttaaaagattttattagtaaacaaactaataaaattaatcacacactttataaagaaattaaaaatatgaataatccttgtaaaactaaactaaaaaatcattttgattcttttaaaagtagttcacctttaaatgatcaatttgatcaattaaaagatcaaatttcttatttaaaatctcaaattcatcaaaataactctaatgatgttaatcagtttttaaaaaatgaagaatttaatagactaaagtctgatattttatctcaaaaacaagaactagaagaatttaaaaaagagctttattttgaaaaaatgcttaatgaaaaactagaaagttttgatagtaaactaaaacaaaaagaaagtattttagaattagaaagactaaaacaagaaattaaagatgaacaaaataaattacgtgaaatgttacttttagaaaaactggttaataactaataaaacgaattttaacttattaggtttataggagcatatttaatttatgcaaaaactctctaataattctaaaataaacaattctactaattctaaactaaaagaattccctttagaaaattaatcagattttgtttaaaaaatctaataaaagaacgtttgttttgcatcttaaacttttctaatattcttatatcgtttttaataggtgttcttttagcttttgttaaaacaggacaaaatagagtgattatcttcaatttttacatactattttttacttgctgtttattatttgtccttattttaaaaatgattcaatttttctttaataaaaatttagaagataagactacctatattgttttaacaaatcaagttagtagaaataaattttttatctcacaatattttttaataattttgattttagcaataaatgttttagtaagttttctacttattaatttagcttatagtgtttttaatagatttaaatacgatatttttattttaaaaatgacattagtatatcttttgtataatttgtttgcctcattttgtctaattaattttataagtatgttaatgtttcttttctctttacaaactacaactataatttgtactttattacttagtttatgttttgtagcaaatataccgatgtcatttgtcagagctaatgaaaaaagttatagtattaaagttttaacacaaataataaccatgaagattttaagttaaatgatgtttatgatacttatacattaaataaaaatatattagagtataagataaatatccttatttatctaaatatatttatcaatattttttaaagaataaatttttaaaagatgatttttcttctaaaagcaatattgatttaagaattaaaatgtgagatgagctaggtctaatcaatcatgaaaaaataataattaatgaaaagaacttaaagttattttcaaaaccttttggaaatagtaaagttcctagttcatggaataggaatgatttattagatttaaaactaattttgaaaaacacatttattacaaataatcaactaaaagaattaatcaaaaaaactactgataaaaataaaaaaaatattttgcttgattttttaaatttttctattgaaattaataattattttgaaaatagcttacaagttaataactatgaattactttatgatttcttgtttttagataatttaaaaaatagtaactatttaacaaaatcaaatgatttaaaatctgttaaatatgaactaaataataaagatattcgaaatatttatgaatatgaacttttaggtgatgctggtgatggttttaaattctctaattcaaaaagtctagtaaataaattaaatttcaatctaatgtatgtagctagaatattagaaaactattttattaaatattctagcaactatattatcctttctacttctcgtgttttaacagatcaattagattgatctagttatattaaaactagaaataaaatgaagtatttttcatatcttaacttgtataacggattatgagttttttatactagtaatttaggtttttattacaaagatatttgatttactccaacttcagattcatttattgaactagaaaatcaaaaaaacttatttttaggttatttagaatatgacctaaagcttttagaaaataattctataagtaaaaatacaactagtaattacactaaaccgcaaatatacttaattactttaattgtaattaatgttttaagttttctcataactttttataaattttaaaaaaaggatttttaaaagaattttttttaaaaaatcatagaaaggaatacaatgaaaggtctattgaagttgttatcgcttgttggtctttcaacagtagtaactagttcagttgttgcatgtgaaaaaacatcagatacaagttctgatcaagatacagataatagtaaatctgataataattaagataagactaaaccagatggtgaaaaataatcgaaacttaattcatagaagctgaaattgcatttaaaaaattattagagaataatttgtcaaaaaacataaattaataggttataaaaaacagtttaattaaaatatcgattaattaaaaaggtttaaaaagaaaggataacaatgaaaaagctactaaaagtattaacagttattggtttttcagcactagctgcagctccagttgttgcatgtgaaaaaacatcagatgataaaacatcaaatcaaaataatagtagtaatcaaaataataactctagataataagcaaaaaaatgagttaattcaaaaatttaataatgaagtaagaaatgtttataatggaacagtaagaccttttattatttctagaacagcatttttatcaaaagattcagatgataaaaatttcttttcaagagaaagtctttataaacttgacaatgaaaaaagtctaaaaagaaatagtgatggatattatagtttctatgaaaatcttgttcaagaaagaaaaaatgagtttgaaaaaactttaagaactgttttagatgttaattctgctgttaacaagtttaaagaaaaaatattaagtttaggtgttgacaaatatcgaactatattaggtggttttaataataataaatgaattaaaggaatcaaatttagattaacagattcaaaaataaagttttttgaagaaaaagattcatttgattcaactattcaagtaggtttagattttcaatatcaatataaagatagtgctgaccaaataaaaattgaaactatttcagatgattttgtaattaatatttctagtgaagaagctgttattaagttagtaaatgatattaaaaaatcttgagttgatcttttattattagatagtaacaatttattaaaagttgattttgaaagattaaaaaaatttttaggtgaaaatcaagtattaactgctcaagacttgttgacaactgcaactaataattacaagagtgtaatatctaaatacaatgaaagtctagctgaagatgttaaagaagaaatttctagacattttattaaaaatagtgaaaataaagtagttaaaaatttattactttcatttaaagatcagaatcaaaaaactgatgttgaaaaaaataatacagaacttaatattggttcattagaaagaagttactataatgctaaaggtaaaaaaactggtactttagaaaaaggttcatatgacttgttgcatttatattttggtgaagtaaagaatgggcaagaaataaacttgcttaacactaaaacaggagatgagaaattagccaaagacttgattaacccttgagtagaaaaaatggctaattataaaactaagtttaaacaaacattagctaatattgctagtggatttgttgatgaagataaactagatgagtttaataataaattagaaaaagataatgttttaaaaaatttatttaaatcatctacttctattgaaagttttgatctaaaaggcttacagttaaaactagaaaatggatatgtacatgatttaggtaatatttcgtttagttattttgtagaacttgataaaaaagataaagagttgaattttgaaaatttagaaaatttaacaagtgaaggttataaaaagtctgctgtatttgatgcatattataaaggaattgaagtaatgctagatcaattccataagttttatggaattcaaaaggcatacccagactatgaaagttcagaacctagctatccactaaaaagactattgtttaatatgacaggtaaaccaagcagtttaaaagatagtgaaaatagtgattttaatatttgagatgaatgagaaaaatatttaagtcaaactgaaaataatagagtagatgattgaactagtttttatagcttagattttaatgctgatgttaaaaaagttaaagaagaatacttgatttctaatatgttaggaattaaaaattttaaagtttttcaagatcaagaacaacaacatcgatatgaacaagaaatttattataaaaatgaaagttatcaaagagactatgagcaaagtaaatt

SEQ ID NO: 4 - protein, 237 aa, Mycoplasma mycoides
MKKLLTILGSIGLIATSGVAVVACNTSDKTKMPNENKGEEKVDLIKVAKQKDLGFISKKDNEIIKKAFIKQNSIDEKKVTVSVKGNGNGVSATGSDNNTTTNTSNGNLNDSAVIELKATNNGNETKTVTVIFEVNNNLEKLIQVKKLKGLADNKDDTILKAIDKLNPKSNLDTSKLSIERKDKKVSIKSSDSTYTGNPVEIEIESKVGVYVGLSLLSVALLASSGFIIYRSLKKKKK

SEQ ID NO: 5 - protein, 646 aa, Mycoplasma mycoides
MGFKSAYISTKEDMVGNVFEKLRANVKMKSDKYSIVVDNFYKKFKDIEIGPFSFNIEKGKITALLGSSGSGKSVFINSLLGTSINYQGNIFINQKERKDSDSIQNNSDIGFYSQMDFSLYSISAYDFLYNMCYVMGLEQKLVKTRLEYWLKKFDLWEYKDKPLKSFSWGMKNRVNLILCFIKEPRILVCDEPGASLDSHWRNQIYKILDEFRRKSNSTIILTVHNINEVYDIIENFVILEKGKLLFCGTKQELNLYKKTKITFENNILLDEVEKILNQNDILTFNLDSDSNSLVIGLKEHQVFSDALNILEKNNFQIKSIASLSINIDAIKKALEDKITKHQPKYQPDINLNSNFNQSISFDKTLNNSNVTNQRDSAFEQLEVEILDNNNNNNNNNNNNNNNNNNNNNNNNNNNNNNNNNIQSVNLINNLLQKTFLNMFNEINNLKQQVNQSNLDNYEFFVDELKAQINSVSEIKKDFLDKIDQLNTDQSSVLDLKDFISKQTNKINHTLYKEIKNMNNPCKTKLKNHFDSFKSSSPLNDQFDQLKDQISYLKSQIHQNNSNDVNQFLKNEEFNRLKSDILSQKQELEEFKKELYFEKMLNEKLESFDSKLKQKESILELERLKQEIKDEQNKLREMLLLEKLVNN

SEQ ID NO: 6 - protein, 195 aa, Mycoplasma mycoides
MFCILNFSNILISFLIGVLLAFVKTGQNRVIIFNFYILFFTCCLLFVLILKMIQFFFNKNLEDKTTYIVLTNQVSRNKFFISQYFLIILILAINVLVSFLLINLAYSVFNRFKYDIFILKMTLVYLLYNLFASFCLINFISMLMFLFSLQTTTIICTLLLSLCFVANIPMSFVRANEKSYSIKVLTQIITMKILS

SEQ ID NO: 7 - protein, 303 aa, Mycoplasma mycoides
MWDELGLINHEKIIINEKNLKLFSKPFGNSKVPSSWNRNDLLDLKLILKNTFITNNQLKELIKKTTDKNKKNILLDFLNFSIEINNYFENSLQVNNYELLYDFLFLDNLKNSNYLTKSNDLKSVKYELNNKDIRNIYEYELLGDAGDGFKFSNSKSLVNKLNFNLMYVARILENYFIKYSSNYIILSTSRVLTDQLDWSSYIKTRNKMKYFSYLNLYNGLWVFYTSNLGFYYKDIWFTPTSDSFIELENQKNLFLGYLEYDLKLLENNSISKNTTSNYTKPQIYLITLIVINVLSFLITFYKF

SEQ ID NO: 8 - protein, 44 aa, Mycoplasma mycoides
MKGLLKLLSLVGLSTVVTSSVVACEKTSDTSSDQDTDNSKSDNN

SEQ ID NO: 9 - protein, 712 aa, Mycoplasma mycoides
MLHVKKHQMIKHQIKIIVVIKIITLDNKQKNELIQKFNNEVRNVYNGTVRPFIISRTAFLSKDSDDKNFFSRESLYKLDNEKSLKRNSDGYYSFYENLVQERKNEFEKTLRTVLDVNSAVNKFKEKILSLGVDKYRTILGGFNNNKWIKGIKFRLTDSKIKFFEEKDSFDSTIQVGLDFQYQYKDSADQIKIETISDDFVINISSEEAVIKLVNDIKKSWVDLLLLDSNNLLKVDFERLKKFLGENQVLTAQDLLTTATNNYKSVISKYNESLAEDVKEEISRHFIKNSENKVVKNLLLSFKDQNQKTDVEKNNTELNIGSLERSYYNAKGKKTGTLEKGSYDLLHLYFGEVKNGQEINLLNTKTGDEKLAKDLINPWVEKMANYKTKFKQTLANIASGFVDEDKLDEFNNKLEKDNVLKNLFKSSTSIESFDLKGLQLKLENGYVHDLGNISFSYFVELDKKDKELNFENLENLTSEGYKKSAVFDAYYKGIEVMLDQFHKFYGIQKAYPDYESSEPSYPLKRLLFNMTGKPSSLKDSENSDFNIWDEWEKYLSQTENNRVDDWTSFYSLDFNADVKKVKEEYLISNMLGIKNFKVFQDQEQQHRYEQEIYYKNESYQRDYEQSKLHVTFPKGIVTEGRRNTHLEFGLLTDLLNFKLKVAEGFTYYTLGAVTIIGKLDDQNGNQDSTKPDGEKDKTKPDGSTEPKQPENQK

SEQ ID NO: 10 - DNA, 6690 bp, Mycoplasma mycoides
aatttgaaggattaggattagcttctttacaaaatattcttactaatatttcaagaatgagaaaccgtacacgtggtaaatacactgctccaatggttattagaacaccaatgggtggaggaattcgtgctttagaacatcatagtgaagctttagaagcagtatatgctcatattccaggagttcaaattgtttgcccatcaactccatacgatacaaaaggattaattttagctgcaattgattcaccagatccagttattgttgttgaaccaacaaaactatatagagcatttaaacaagaagttccagatgaacactacatagtaccaattggagaagcttataaaattcaagaaggtaatgatttaactgttgttatttatggtgctcaaactgttgattgtcaaaaagctattgcgttattaaaagaaactcatccaaacgcaactattgatttaattgatttacgttctattaaaccatgagataaaaaaatggtagttgaatcagttaaaaaaacaggaagattattagttgttcatgaagctgttaaatcattctcagtttcagctgaaatcattacaactgttaatgaagaatgttttgaatacataaaagcacctttatcaaggtgtacaggttatgatgttattactccatttgatagaggagaaggttacttccaagttaaccctaaaaaagttctagtcaaaatgcaagaattgttagactttaaattttaaataataaatatttagaaaggaaaaagatatgttcaaagttaaatttgctgacataggtgaaggtctaacagaaggaatagtcgctgaagttttagttaaagttggtgatgttgttaaagaaggacaatcattatactttgttgaaactgataaagtaaacagtgaaatacctgctccagtggctggaaaaattgcagtaattaacattaaagctggacaagaaatcaaagttggtgatgtagttatggaaattgatgaaggaacaggtgcatctgtagctagtgaaccaaaagctgaagctaaatcagaagctaaagttgaagtagttgaagaaaatgctagtgtagttggtgctactccagtttcaaatgatttaattgttagaaaacaagcatctacagttgcaaaatcaagcactattaaagctacacctctagcaagaaaagttgctgctgatctaaatgttgatttatctttagtaactccaacaggaccaaaccaaagaattttagttgctgatattaaaaactatcattcttcatcagctcaaccagttagtcaaccagctccagctccagttgtaactccaacaattaaagttgttgaaccaagtgcacctttatcttgagatgaagttccaatgaatggtgttagaaaagctacagtaaaagcaatgacaaaatcacatactgaaattgctgcatttactggtatgaaaaacactgatattactgaaactcacaaaatgagaactgaattaaaagatcatgctgcagctagtggaattaaattaacttacttagcatttattattaaagctgttgctaaatcattacgtgatatgccaaatattaatgtaagaggtgattttgcaaacaacaaaatccaatttatgcacaacattaatattggaattgcagtagatacaccaaacggattaatggttccagttattaaaggtgttgatcacttaagtgtatttgaaattgcaatcaaaattaatgaattagcaaacaaagctaaagatggtaaattaacaagagctgaaatgactgaagcaacatttactgtttcaaactttggttcagtagaactagattatgctactcctattattaactcaccagaatcagctattttaggagttggtgcaatgtctcaaactcctttatatatcaatggtgaattacaaaaaagatttataatgccattatcaatgacttgtgatcatagaatcattgatggtgcagatgctggaagatttttaattaaagtacaagattatctatcaaaaccagtactattgtttatgtaattagtgaagggataaaatatgttcaaagttaaatttgctgacataggtgaaggtttaacagaaggaacagtcgctgaagttttagttaaagttggtgatgttgttaaagaaggacaacctttatattttgttgaaaccgataaagtaaatagtgaaattccttctccagttgctggaaaaattgcaataataaatatttctactggtcaagaaattaaagttggtgatgtagttattgaaattgatgatggaacatcagtttctacaagcgaacctaaagttgaagtagttgaagaaaatgctagtgtagttggtgctactccagtttcaaatgatgttttaccaagtagagcaccaaaaccaaaacctgaagctccaaaagtggatgttcaaattgaagatacatttgatgtttgtgtagttggtgcaggaattggtggttatgttactgctattaaatctgctcaattaggtctaaaaactttaattatcgaaaaagaatattatggtggagtttgtttaaatgttggatgtattcctacaaaaactttattaaaaacttctcatgtttatcacgatataatgcataaagcaaaagaattaggaattgttttacaaaatactgaaaaagtagtgattgattgagctcaagtacttcaaagaaaaaatggtgttgttaagaaattaacaggtggagttaaatatctactagataaaaataaagtaactcaaatcaaaggtgaagctgttgctttagataaaaatacaatttcagtaaataataaaaattatcgtgttaataacttaattattgcttctggatcaacaccaaatcatttaccacttccaggatttgatcaaggaagaaaagatggaatcatcattgattcaactggaattttatcagttccaaaaattcctgagactttagttgtaattggtggaggggttattggtattgagtttagttgtttatttgctagtcttggtacaaaagttactgttttacaaggattaccaactgttttagaaatgctagataaagatattattgatgctatgactaaagagttgaaaaacagatacaacattgaagttattacaaatgcttcagttaaagaatttaaaaatggatctgtagtttatgaaattgatgataaagatcaaatgattaaaggagaatacgttttagaatcagttggacgtaaaacttcaataactggatttgaaaacatcggtttagaactaactccaagaaaagctattgttgttaatgaatatcaagaaactaatttagatggtgtttatgcaattggtgatgttgttggaaaagtaatgttagctcatactgcagttaaaggagctattgttgctgctaatagaattgctaaaaaagctaataaagatcatgctgaagatattgttatggattattacagaattccatcatgtatttatacacatccagaagtttcaatgataggaaaaactgaacaacaattaaaacaagaaaatattgaatacaaaacctttaaattcccattctcagctattggtaaagctttagctgataatgatacttcaggatttgtaaaaattattatagaacctaaatacaaaactattttaggtgcacatattattggaaatagagcaactgaaatgatttctgaaattgctgctgttattgagtgtgaaggaacaattactgaaattgctaatacaattcaccctcacccaacaatgagtgaagcaattggagaagcagcagaagctctagaaacaggaaaagctattcatttttaattaatttatagaaagaataatatgtatacattagaagaaattaaaaatcaactagttttaaaatcagaaaaaaaatctattgtttttccagaaggtgaatctgaaattattcaatctgtggctaaaactttagttgatgaaaaattaggattaccaattctattatttaaatcttcaaaagaagttccaagtgaaattaaaaataattcatcaattaaaactatttgtttagatgaatttgacacaaaagaatttagtgaagaatttgtaaaacttagaaaaggtaaagcaactattgaagttgcacatcaagtaatgcaattaccaaattatgttggagctatgctagttaaattaaatcaagctgattgtatgttatctggattaaacaatacaacagctgatacaatcagaccagctttacaaattattggtacaaaacctggatataatattgcaagtagtatctttattatgtcaaaaggtgatgaaaattacatttttactgattgtgctttaaacattagaccaacaagcgaacaattagttgaaatcacacaaatggcagttgattttgcaaagactttaaatgtaaaaaatgttgaagctgctttattaagctattcaacaaaaggtagtggtaaaggtgaagatgttgatagagttcacaaagcagttgaaattttaaaatctagtcaaaatgattatgtttgtgaaggtgaaattcaatttgatgctgcttttgataaaaaaactagagataaaaaattcaaaaactgtttattaactaaacaaactccagatatctttgtttttccagatataaatgctggaaatattggttataaaattgctcaaagaatgggtggttttgaagcaattggaccttttgttttaggattaaatcaaccaattaatgatttgagtagaggtgctacatttatagatgttttaaatacagctataatgacactacacttatcttattaaggagaaaattaaatgattttagtaattaattctggaagtagttcaattaaatttaaattatttgatacttcaaaaacaattgaaccaatattagatggtctagcagaacgtattgggattgatggatttttaaaatttgaacacaataatcaaaaatataaatttgaagatcctcttccagatcatgaacatgctattcaattaattttaaataaattacttgagttaaaaattatatcaaatattgatgaaattaatggagttggatttagagtagttcatggtggtgaaatttcacattcatcaattattactgatgaaatactatcaaaaattcaagatagtgttaaattagctccacttcataatccagctgcaattattgctataaaagctgttaaaaaattaatgccaaatactagtatggtagcttgttttgatacagcatttcatcaaactatgccagaagtaaattatttatacactgttccttataaatgatatgaagaatttggtgtaagaaaatatggatttcatggaattagttatgaatacatagttaataaatcttctgaaattttaaataagaaaaaagaaaatttaaacttaattgtttgtcatttaggaaatggtgcaagtatttcatgtattaaagatggtaaatcttatgatacttcaatgggattaactccattagctggtttaatgatgggaactagaagtggagatattgatgtatcaatatgtgagtatattgcaaaacaaacaaatactgacatcttttcaattactcaaactttaaataaacaatcaggacttttaggtttaagtcaagtttcagctgacatgagagatgttttagaacaatatgatagaaatgataaaaaagcagttgttgctgttgaaaaatatgttcaaattgttgctgattttatagttaagtatgcaaattatttagataacattgatgcagtagtatttacagcaggaattggtgaaaacgctgatgttattagagatttaatttgtaaaaaagttaaacttttaaatttacaaattgatcaagataaaaaccaagctaaatactcagattataaattaatttctagtgaaaaatcaaaaattccagtttatgctattagaacaaatgaagaaaaaatgatttgtttagatactttaaacttaattaaataaaagatataataattaaaacacttagcttaaaaagctaagtgtttactatttaataaaagaaagaaatcaatgaaaaaatcattaaaatatttaagtagtcttagtttaattttagttccattagttactatttcatgtactcatactaataaaattaaacctggttttattccaactattaattcatctaataaaaaaaacataattgatttaagtaataaattagaagaatttaaatctattttagaaaataatcctagaattgataaaaatttaaaagataaaattgttaaaaaattaacagaaattaatacaaaatcaataagtttaaataatttggctaatataaaaaacattttatttggacttaaaaatttttctaataattttaatagtgataatcttttaagttttttaaaaaaggttaaagaattattattagaactagaaattaatgaattagtaaatgaagttgatcaacatattaataaaataactaatgataagcaaaataatcaaccaattataaatgctgatagtaatgttgaagacaagtttatagaaggctctgtttatagtcctgcaaatcatagttatccagaatttgcaaataagtttactccagttccagcagaagaa

SEQ ID NO: 11 - DNA, 1121 bp, Artificial
  Lambda-B1 clone sequence
ggnnngggggnnntttannttngttnagttnattngccctttgcaaacnccccatnncgattttagaggatcccccgaatttgaaggattaggattagcttctttacaaaatattcttnttaatatttcaagaatgagaaacngncangtggtaaatacactgctccaatggttattagaacaccaatgggtggaggaattcgtgctttagaacatcatagtgaagctttagaagcagtatatgctcatattccaggagttcaaattgtttgcccatcaactccatacgatacaaaaggattaattttagctgcaattgattcaccagatccagttattgttgttgaaccaacaaaactatatagagcatttaaacaagaagttccagatgaacactacatagtaccaattggagaagcttataaaattcaagaaggtaatgatttaactgttgttacttatggtgctcaaactgttgattgtcaaaaagctattgcgttattaaaagaaactcatccaaacgcaactattgatttaattgatttaccgttctattaaaccatgagataaaaaaatggtngttgaatcagttaaaaaaacaggaagattattagttgttcatgaagctgttaaatcattctcagtttcagctgaaatcattacaactgttaatgaagaatgttttgaatacataaaagccctttatcaaggtgtcagggtatgatgttattactccatttgatagaggagaaggttntttncaggtaacccctaaaaaagttctagtnaaatgcaagaattgttagactttaaatttaaataataaanttttagaaaggaaaaagatttgtcaaagttaatttgctgcataggtgaaggtntacaanaanggaatagccctgaagttttattaaaggtnggnggtgttgtaaagaaggacattcnttnttctttgttgaactggataaagtaaaacagggnaattcctnctccagnggttggaaaaattgggttattttaannttaaagntnggcaaaaaantnaaagttggnggtttgnnttttgggaaattggtgaaggaacanggnctntttgtggtttgggaaccaaaagttgagtt

SEQ ID NO: 12 - DNA, 1101 bp, Artificial
  Lambda-B1 clone sequence
aaaattnttnanttcnaatgccccttntgnaaanccccgctaccntttggnnaaaggganccccgatgggcccgcggccgctctagaagtactctcgagaagctttttgaatttcttctgctggaactggnggnagnggtatttgcaaattctggataactatgatttgcaggactataaacagagccttctataaacttgtcttcaacattactatcagcatttataattggttgattattttgcttatcattagttattttattaatatgttgatcaacttcatttactaattcattaatttctagttctaataataattctttaaccttttttaaaaaacttaaaagattatcactattaaaattattagaaaaatttttaagtccaaataaaatgttttttatattagccaaattatttaaacttattgattttgtattaatttctgttaattttttaacaattttatcttttaaatttttatcaattctaggattattttctaaaatngatttaaattcttctaatttattacttaaatcaattatgnttttttattagatgaattaatagttggaataaaaccaggttaattttattagtatgagtcatgaaatagtanctaatggaactaaaaattaaacctaagactcttaaaatatttaaangattttttcatggatttctttctttnattaaaaggnaaaccactaancttttaagctaaagggggtaaataattataaccttnanttaaataaagtttaaagggntctaaaacaaaatnattttttcttcatttgggcnnaaancataaaccngggaattttggtttttccctagaaaataaattataancccgnggtntnagnttggggttttccttgggcnaattggnnaatttnaaaaggttaaccnttttaccaannnaaactctaaaancaaagcgggttccccaatncngggngggaaaacnnccgncccaagggtntcnaaaaattngnctacctcacnntaaanncgnccatttggnanatttttnaaccncaccncngggtttntnnttccanncnngggcgnaaaanaccnctngggccg

SEQ ID NO: 13 - protein, 329 aa, Mycoplasma mycoides
MAIINNIKAVTDALDCAMQRDPNVIVFGEDVGTEGGVFRATQGLAVKFGNDRCFNAPISEAMFAGVGLGMAMNGMKPVVEMQFEGLGLASLQNILTNISRMRNRTRGKYTAPMVIRTPMGGGIRALEHHSEALEAVYAHIPGVQIVCPSTPYDTKGLILAAIDSPDPVIVVEPTKLYRAFKQEVPDEHYIVPIGEAYKIQEGNDLTVVIYGAQTVDCQKAIALLKETHPNATIDLIDLRSIKPWDKKMVVESVKKTGRLLVVHEAVKSFSVSAEIITTVNEECFEYIKAPLSRCTGYDVITPFDRGEGYFQVNPKKVLVKMQELLDFKF

SEQ ID NO: 14 - protein, 428 aa, Mycoplasma mycoides
MFKVKFADIGEGLTEGIVAEVLVKVGDVVKEGQSLYFVETDKVNSEIPAPVAGKIAVINIKAGQEIKVGDVVMEIDEGTGASVASEPKAEAKSEAKVEVVEENASVVGATPVSNDLIVRKQASTVAKSSTIKATPLARKVAADLNVDLSLVTPTGPNQRILVADIKNYHSSSAQPVSQPAPAPVVTPTIKVVEPSAPLSWDEVPMNGVRKATVKAMTKSHTEIAAFTGMKNTDITETHKMRTELKDHAAASGIKLTYLAFIIKAVAKSLRDMPNINVRGDFANNKIQFMHNINIGIAVDTPNGLMVPVIKGVDHLSVFEIAIKINELANKAKDGKLTRAEMTEATFTVSNFGSVELDYATPIINSPESAILGVGAMSQTPLYINGELQKRFIMPLSMTCDHRIIDGADAGRFLIKVQDYLSKPVLLFM

SEQ ID NO: 15 - protein, 595 aa, Mycoplasma mycoides
MFKVKFADIGEGLTEGTVAEVLVKVGDVVKEGQPLYFVETDKVNSEIPSPVAGKIAIINISTGQEIKVGDVVIEIDDGTSVSTSEPKVEVVEENASVVGATPVSNDVLPSRAPKPKPEAPKVDVQIEDTFDVCVVGAGIGGYVTAIKSAQLGLKTLIIEKEYYGGVCLNVGCIPTKTLLKTSHVYHDIMHKAKELGIVLQNTEKVVIDWAQVLQRKNGVVKKLTGGVKYLLDKNKVTQIKGEAVALDKNTISVNNKNYRVNNLIIASGSTPNHLPLPGFDQGRKDGIIIDSTGILSVPKIPETLVVIGGGVIGIEFSCLFASLGTKVTVLQGLPTVLEMLDKDIIDAMTKELKNRYNIEVITNASVKEFKNGSVVYEIDDKDQMIKGEYVLESVGRKTSITGFENIGLELTPRKAIVVNEYQETNLDGVYAIGDVVGKVMLAHTAVKGAIVAANRIAKKANKDHAEDIVMDYYRIPSCIYTHPEVSMIGKTEQQLKQENIEYKTFKFPFSAIGKALADNDTSGFVKIIIEPKYKTILGAHIIGNRATEMISEIAAVIECEGTITEIANTIHPHPTMSEAIGEAAEALETGKAIHF

SEQ ID NO: 16 - protein, 322 aa, Mycoplasma mycoides
MYTLEEIKNQLVLKSEKKSIVFPEGESEIIQSVAKTLVDEKLGLPILLFKSSKEVPSEIKNNSSIKTICLDEFDTKEFSEEFVKLRKGKATIEVAHQVMQLPNYVGAMLVKLNQADCMLSGLNNTTADTIRPALQIIGTKPGYNIASSIFIMSKGDENYIFTDCALNIRPTSEQLVEITQMAVDFAKTLNVKNVEAALLSYSTKGSGKGEDVDRVHKAVEILKSSQNDYVCEGEIQFDAAFDKKTRDKKFKNCLLTKQTPDIFVFPDINAGNIGYKIAQRMGGFEAIGPFVLGLNQPINDLSRGATFIDVLNTAIMTLHLSY

SEQ ID NO: 17 - protein, 393 aa, Mycoplasma mycoides
MILVINSGSSSIKFKLFDTSKTIEPILDGLAERIGIDGFLKFEHNNQKYKFEDPLPDHEHAIQLILNKLLELKIISNIDEINGVGFRVVHGGEISHSSIITDEILSKIQDSVKLAPLHNPAAIIAIKAVKKLMPNTSMVACFDTAFHQTMPEVNYLYTVPYKWYEEFGVRKYGFHGISYEYIVNKSSEILNKKKENLNLIVCHLGNGASISCIKDGKSYDTSMGLTPLAGLMMGTRSGDIDVSICEYIAKQTNTDIFSITQTLNKQSGLLGLSQVSADMRDVLEQYDRNDKKAVVAVEKYVQIVADFIVKYANYLDNIDAVVFTAGIGENADVIRDLICKKVKLLNLQIDQDKNQAKYSDYKLISSEKSKIPVYAIRTNEEKMICLDTLNLIK

SEQ ID NO: 18 - protein, 651 aa, Mycoplasma mycoides
MKKSLKYLSSLSLILVPLVTISCTHTNKIKPGFIPTINSSNKKNIIDLSNKLEEFKSILENNPRIDKNLKDKIVKKLTEINTKSISLNNLANIKNILFGLKNFSNNFNSDNLLSFLKKVKELLLELEINELVNEVDQHINKITNDKQNNQPIINADSNVEDKFIEGSVYSPANHSYPEFANKFTPVPAEEIYKELYDRTFSIKFLTRLNNGDSLSNGTGTGWLLDYHKYNNENKYKLFLATNLHVLSEFSNSLTEEQNKEFNYYEPSNNKVIAIGLGKAENVNDFSSKNNKKDFQNKISKYYLSNQDFKNYSQIDYSNSNSELTTAISEPKIVFGAVDFMNRKAVEKYDDAIKQSAEEYYKYKKDKLNDNEKIAWEDYFKTKNIPIMVDFAVFEIDVDLNKTDNTLKSWINDAIKGLDKYLERLKNTNNLPNQDKNISNYLQSKDYISALFTKDNNSNNLYNAKNIYIAGYPVNNKTTYWMKNNPTERSSDKSDIDWRTSKSNREIFGFANEFQSSISIVPNFRIFDNYWHRVFATFYGYQYNVNFSSLYYGASGSLAYNEFGQMIGIYNNVRSNVEFGDLLQHATIAPFLQSDNIKIGDNIIYAYNLIDGTDKTRYKYQKSSFRENLQKLYPNGFSDNSKSTKLFDNIFN